SEQUENCE LISTING (4 sequences)

SEQ ID NO: 1 - DNA, 22 bp, synthetic construct
  source 1..22: mol_type = other DNA
ctgaattggtcatcccaggtaa

SEQ ID NO: 2 - DNA, 22 bp, synthetic construct
  source 1..22: mol_type = other DNA
gataggcagacatcatgctagg

SEQ ID NO: 3 - DNA, 23 bp, synthetic construct
  source 1..23: mol_type = other DNA
gctatgagtcaaggagtgtaacc

SEQ ID NO: 4 - DNA, 21 bp, synthetic construct
  source 1..21: mol_type = other DNA
cacaaagatggctgaggaaga

(19) United States
(12) Patent Application Publication
(10) Pub. No.: US 2025/0144128 A1
(43) Pub. Date: May 8, 2025

(54) CANCER TREATMENT

(71) Applicant: Nucana plc, Edinburgh, Central Scotland (GB)

(72) Inventors: David James Harrison, Edinburgh, Central Scotland (GB); Mustafa Elshani, Edinburgh, Central Scotland (GB)

(73) Assignee: Nucana plc, Edinburgh, Central Scotland, GB (GB)

(21) Appl. No.: 18/943,650

(22) Filed: Nov. 11, 2024

Related U.S. Application Data
(63) Continuation of application No. PCT/GB2023/051245, filed on May 12, 2023.

(30) Foreign Application Priority Data
May 12, 2022 (GB) 2206948.8
Nov. 18, 2022 (GB) 2217305.8

(51) Int. Cl. A61K 31/7076; A61P 35/00; A61P 37/04; C07K 16/28

(52) U.S. Cl. CPC A61K 31/7076; A61P 35/00; A61P 37/04; C07K 16/2818

(57) ABSTRACT

The invention provides NUC-7738 (3′-deoxyadenosine-5′-O-[phenyl(benzyloxy-L-alaninyl)] phosphate), a phosphoramidate derivative of 3′-deoxyadenosine, for use in the treatment of a proliferative disease, such as cancer, by reducing the amount of extra-cellular PD-L1 protein (e.g. soluble PD-L1 or exosomal PD L1) and/or increasing the amount of OX40-L. The invention also relates to the use of NUC-7738 in combination with an immune oncology agent such as an immune checkpoint inhibitor.

### CROSS-REFERENCE TO RELATED APPLICATIONS

This application is a continuation of International Patent Application No. PCT/GB2023/051245, filed on May 12, 2023, which claims priority to Great Britain Patent Application No. 2206948.8, filed on May 12, 2022; and Great Britain Patent Application No. 2217305.8, filed on Nov. 18, 2022. The entirety of each of these applications is incorporated by reference in their entireties for all purposes.

### INCORPORATION BY REFERENCE

The contents of the XML file named “16080-340WO1US1_2024-11-11 XML FOR FILING.xml”, which was created on Nov. 4, 2024, and is 5 KB in size, are hereby incorporated by reference in their entireties.

### FIELD OF THE INVENTION

The invention relates to medical uses and methods for treating a proliferative disease such as cancer by reducing the amount of extra-cellular PD-L1 protein, such as soluble PD-L1 and/or exosomal PD-L1 protein, produced by the proliferative disease (e.g. cancer) cells and/or increasing the amount of OX40L produced by said cells. The inventors have discovered that the protide molecule NUC-7738 reduces the amount of soluble PD-L1 and/or exosomal PD-L1 protein produced by cancer cells and increases the amount of OX40L produced by cancer cells. This observation offers up new clinical opportunities for NUC-7738 in treating cancer. The present invention also relates to the use of NUC-7738 in combination with an immune checkpoint inhibitor (ICI), such as PD-1 or PD-L1 inhibitor, to treat proliferative diseases like cancers, such as melanoma and lung cancer. Because of its ability of reducing the amount of PD-L1 protein produced by a cancer cell, it opens up the ability for the NUC-7738/immune checkpoint inhibitor combination to be used in patients that have become resistant to immune checkpoint blockade, in patients that are refractory to immune checkpoint blockade, in patients who have relapsed following immune checkpoint inhibitor therapy or in patients who may not be considered for immune checkpoint inhibitor therapy due to concerns that they would not respond to therapy. Suitably this is possible because the combination with NUC-7738 is expected to allow the use of a lower dose of immune checkpoint inhibitor.

### INTRODUCTION

3′-deoxyadenosine (3′-dA) is a nucleoside analogue of adenosine that lacks the 3′-hydroxyl group on the ribose moiety at the 3′ position and can be produced synthetically from adenosine. Reference for such synthetic procedures is made to Robins, J. R. et al J. Org. Chem. 1995, 60, 7902-7908 and Aman, S. et al Organic Process Research & Development 2000, 4, 601-605.

3′-dA has been studied extensively as an anti-cancer agent but, despite showing potent anti-cancer activity in preclinical studies it has not been successfully developed or approved as an anti-cancer agent.

Because of its structure, 3′-dA and its phosphorylated forms (3′-dA-mono, di and tri-phosphate) could potentially interfere with any process respectively requiring adenosine or adenosine mono, di or tri-phosphate (AMP, ADP or ATP). However, after administration, 3′-dA is quickly deaminated by adenosine deaminase (ADA), and rapidly metabolized to an inactive metabolite, 3′-deoxyinosine, in vivo. Reference is made to Tsai, Y-J et al J. Agri. Food Chem. 58 4638-43 (2010).

NUC-7738 (3′-deoxyadenosine-5′-O-[phenyl(benzyloxy-L-alaninyl)] phosphate) is a phosphoramidate transformation of the monophosphate of the nucleoside 3′-dA. Unlike 3′-dA, it is capable of transporter-independent entry into the cell and 3′-dAMP is generated independently of the activating enzyme adenosine kinase (AK), a process that has been shown to be rate-limiting during the formation of the phosphorylated forms of 3′-dA. There is also no breakdown by the enzyme adenosine deaminase (ADA), thus resulting in higher intracellular levels of 3′-dATP. NUC-7738 thus can bypass mechanisms involving transport, activation and breakdown that limit the utility of 3′-dA.

Checkpoint inhibitors are a form of cancer immunotherapy that target immune checkpoints, key regulators of the immune system that, when stimulated can dampen the immune response to cancer. These immune checkpoints have co-evolved with stimulatory immune receptors as a way of controlling immune responses and preventing excessive immune reactions which could be dangerous. Cytotoxic T-cells express the immune checkpoint protein PD-1 on their cell surface. If this binds to its ligand, PD-L1, this signals to the T-cell that it should not be activated, even if the T-cell has recognised something that it normally would kill.

In addition to PD-1 and PD-L1, other proteins expressed by immune cells and non-immune cells (including cancerous cells) are part of immune checkpoint signalling pathways including CTLA-4, LAG-3, TIGIT, BTLA, OX40/OX40L and TIM-3. Cancers can manipulate this system to protect themselves from immune attack through the expression of proteins that are part of immune checkpoint signalling pathways.

Checkpoint inhibitor therapy can block the inhibitory checkpoints, restoring immune system function and resulting in immune-mediated cancer cell death. Current approaches focus on use of a PD-1/PD-L1 inhibitor as monotherapy or of a PD-1/PD-L1 inhibitor in combination with a checkpoint inhibitor targeting another part of the checkpoint signalling pathway (e.g. a CTLA-4 inhibitor). Checkpoint inhibitor therapy can also be given in combination with chemotherapy or targeted therapy.

Resistance to checkpoint inhibitors can either exist prior to treatment with checkpoint inhibitor therapy (primary resistance) or can develop following treatment (acquired resistance). Patients with primary resistance do not respond to initial therapy and their cancer continues to grow, whereas patients with acquired resistance initially respond to therapy but their disease subsequently progresses as the resistance mechanisms take effect.

PD-L1 is expressed on the surface of tumour cells, immune cells and other cells in the tumour microenvironment, but it is also released from tumour cells and is present in several extra-cellular forms (see Daassi et al. “The importance of exosomal PDLI in tumour immune evasion”. Nature Reviews Immunology 20 (4): 209-215, 2020). This includes being found as a soluble protein (soluble PD-L1 or sPD-L1) in plasma or associated with exosomes, a type of extra-cellular vesicle (exosomal PD-L1 or xPD-L1). Exosomes are a form of extra-cellular vesicle, produced by many cell types, particularly in sites of inflammation or cancer. They are characterised by their size and shape, but also by expression of CD81 on their surface. They are important in conveying messages between cells. Several studies have shown that soluble and exosomal PD-L1 can act as decoys, effectively neutralising the benefit of anti-PD-L1 immune checkpoint therapy (see Zhang et al. Cell Biosci 9:19, 2019). Soluble PD-L1 is circulating programmed-death ligand 1 that is measurable in the serum of patients with various types of cancer Finkelmeier et al. “High levels of the soluble programmed death-ligand (sPD-L1) identify hepatocellular carcinoma patients with a poor prognosis”. Eur J Cancer. 59:152-9, 2016; Okuma et al. “High plasma levels of soluble programmed cell death ligand 1 are prognostic for reduced survival in advanced lung cancer”. Lung Cancer. 104:1-6, 2017; Chang et al. “The correlation and prognostic value of serum levels of soluble programmed death protein 1 (sPD-1) and soluble programmed death-ligand 1 (sPD-L1) in patients with hepatocellular carcinoma.”. Cancer Immunol Immunother. 68:353-63, 2019; Shigemori et al. “Soluble PD-L1 expression in circulation as a predictive marker for recurrence and prognosis in gastric cancer: direct comparison of the clinical burden between tissue and serum PD-L1 expression”. Ann Surg Oncol. 26:876-83, 2019). A previous study has shown that sPD-L1 may impair host immunity and contribute to systemic immunosuppression, subsequently leading to cancer progression and a poor clinical outcome (Frigola et al. Clin Cancer Res. (2011) 17:1915-23, 2011). PD-L1 expressed on or released from cancer cells signals to cytotoxic T-cells that are in close proximity to them to deactivate them. Reducing exosomal PD-L1 release from tumours, or reducing the amount of soluble PD-L1, is therefore a novel potential target for therapy to accompany immune checkpoint blockade.

PD-1 (also known as programmed cell death protein 1 and CD279) is a checkpoint protein on T cells, which acts as a type of “off switch” to assist in preventing the T cells from attacking normal healthy cells in the body. It does this when it binds to PD-L1 (also known as programmed death-ligand 1 and CD274)], a protein on some normal and cancer cells. When PD-1 binds to PD-L1 on a cell, it induces a signal that instructs the T cell to leave the other cell alone. Some cancer cells produce large amounts of PD-L1, which helps them avoid identification by the adaptive immune system and thus evade attack from the host immune system.

Immune checkpoint proteins present on immune cells and/or cancer cells [e.g. CTLA4 (also known as cytotoxic T-lymphocyte-associated protein 4 and CD152), LAG3 (also known as lymphocyte-activation gene 3 and CD223), PD-1 and PD-L1, TIGIT, TIM-3, and BTLA] are molecular targets that have been found to play an important role in regulating anti-tumour immune responses. Inhibitors of these immune checkpoint proteins (e.g. CTLA4, LAG3, TIGIT, TIM-3, BTLA, PD-1 and/or PD-L1 inhibitors) promote an anti-tumour immune response that can be utilised to effectively treat certain forms of cancer.

OX40L, also known as TNFSF4 and CD252, is expressed on antigen presenting cells but has been described in many other cells including melanoma cells. It binds to OX40, a member of the TNF Receptor superfamily, on T lymphoctes to promote activation of effector T cells. There are several clinical studies underway investigating the possible role of OX40L as a way of enhancing anti-neoplastic cell T lymphocyte responses, including in combination with drugs that block the PD-L1/PD-1 axis.

Ligation of OX40L with OX40 in T-lymphocytes promotes maintenance and generation of memory CD8+T cells. Furthermore, it has been shown that induced expression of OX40L in tumours led induction of antitumour immunity (Andarini, Sita, et al. “Adenovirus vector-mediated in vivo gene transfer of OX40 ligand to tumor cells enhances antitumor immunity of tumor-bearing hosts.” Cancer Research 64 (9): 3281-3287, 2004).

### SUMMARY OF THE INVENTION

Data is presented in the example section herein that shows that the NUC-7738 causes a reduction in mRNA and protein of extra-cellular forms of PD-L1, in particular soluble and exosomal PD-L1. PD-L1 produced by cancer cells is known to be important in aiding the cancer cell to avoid host immune attack. Soluble PD-L1 and exosomal PD-L1 (types of extra cellular PD-L1) can act as a decoy protein blocking activation of T-cells and reducing their capacity to kill tumour cells. Accordingly, NUC-7738 can be used to reduce the production of this decoy protein rendering the cancer cell more susceptible to attack via the host's immune system. Data is presented in the example section that shows that NUC-7738 also causes an increase in the amount of OX40L produced by cancer cells. By reducing the amount of extra cellular PD-L1 (e.g soluble PD-L1 and/or exosomal PD-L1), and/or increasing the amount of OX40L produced by the cancer cells, NUC-7738 is exhibiting the properties of an immune sensitiser and so can be used alone or to potentiate the therapeutic effect of oncology agents or therapies, which can include immune checkpoint inhibitors (e.g. CTLA4, LAG3, PD-1 or PD-L1 inhibitors), as well as cancer vaccines and adoptive cell therapies such as chimeric antigen receptor T cells (CAR-T), tumor-infiltrating lymphocyte (TIL) and natural killer (NK) cell therapies. NUC-7738 may also be suitable for treating cancer patients with high levels of soluble PD-L1 and/or exosomal PD-L1 in the plasma and/or with tumours expressing low levels of OX40L and for use in treating cancers that have developed resistance to certain immune checkpoint inhibitors. Furthermore, by reducing the amount of the decoy protein PD-L1, it may allow the use of lower doses of an immune checkpoint inhibitor, and so not only reduce the toxic side effects of such agent but allow patients that have had to cease treatment with an immune checkpoint inhibitor, for example due to toxicity, to reinstate treatment with the or an immune checkpoint inhibitor but with a lower less toxic dose. NUC-7738 is therefore suitable for use in combination with an immune checkpoint inhibitor in the treatment of a cancer patient that has had their treatment with an immune checkpoint inhibitor stopped, perhaps for toxicity reasons.

Thus, the present disclosure relates, in part, to methods for treating a proliferative disease such as cancer. Suitably the method comprises administering a therapeutically effective amount of a composition to a subject in need of such composition, wherein said composition comprises NUC-7738. Suitably, the therapeutically effective amount is an amount capable of reducing the production of extra-cellular PD-L1 (such as soluble PD-L1 and/or exosomal PD-L1) by the cancer cells and/or increasing the production of OX40-L by the cancer cells. In particular embodiments, the NUC-7738 is capable of reducing the levels of soluble PD-L1 produced by cancer cells. In particular embodiments, the NUC-7738 is capable of reducing the levels of exosomal PD-L1 produced by cancer cells. In particular embodiments, the NUC-7738 is capable of increasing the production of OX40-L by the cancer cells. Suitably, the NUC-7738 enhances the host's adaptive immune system. In particular embodiments, the NUC-7738 is acting as an immune sensitiser. In particular embodiments, the NUC-7738 can be used to potentiate the effects of immunotherapy. Suitably the immunotherapy is an immune checkpoint inhibitor or a cancer vaccine or an adoptive cell therapy (also known as cellular immunotherapy) such as CAR-T cell therapy. In particular embodiments, the NUC-7738 is used in combination with an immune checkpoint inhibitor. In particular embodiments the NUC-7738 is used in combination with an immune checkpoint inhibitor selected from pembrolizumab, cemiplimab, dostarlimab, nivolumab, durvalumab, ipilimumab, atezolizumab, and avelumab. In particular embodiments, the NUC-7738 is used in combination with an an anti-PD-1 antibody selected from pembrolizumab, cemiplimab, dostarlimab and nivolumab. In particular embodiments, the NUC-7738 is used in combination with pembrolizumab to treat cancer, such as cutaneous melanoma.

In various embodiments, the proliferative disease is one in which high levels of extra-cellular PD-L1 (e.g. soluble PD-L1 and/or exosomal PD-L1) contribute to the pathogenesis of the proliferative disease, e.g. cancer. In particular embodiments, high levels of soluble PD-L1 and/or exosomal PD-L1 inhibits or blocks the host immune system from attacking the cancer cell. In particular embodiments, high levels of soluble PD-L1 and/or exosomal PD-L1 inhibits or blocks the effect of an immune checkpoint inhibitor given to the patient. Accordingly, in particular embodiments, NUC-7738 is for use in treating a patient whose cancer cells express high levels of extra-cellular PD-L1 (such as soluble PD-L1 and/or exosomal PD-L1) protein.

In particular embodiments, the NUC-7738 reduces the expression of soluble PD-L1 and/or exosomal PD-L1 protein. In particular embodiments, the NUC-7738 reduces the transcription of the mRNA encoding soluble PD-L1 and/or exosomal PD-L1 protein.

In various embodiments, the proliferative disease is one in which low levels of OX40-L contribute to the pathogenesis of the proliferative disease, e.g. cancer. In particular embodiments, low levels of OX40-L inhibit or blocks the host immune system from attacking the diseased cell.

In particular embodiments, low levels of OX40-L inhibit or blocks the effect of an immune checkpoint inhibitor given to the patient. Accordingly, in particular embodiments, NUC-7738 is for use in treating a patient whose cancer cells express low levels of OX40-L protein.

In particular embodiments, the NUC-7738 increases the expression of OX40-L protein by the cancer cells. In particular embodiments, the NUC-7738 increases the translation of the mRNA encoding OX40-L.

According to one aspect the present invention provides NUC-7738 for use in the treatment of a proliferative disease, such as cancer by reducing the amount of extra-cellular PD-L1 protein and/or increasing the amount of OX40-L protein produced by the diseased cells. Suitably, the extra-cellular PD-L1 is soluble PD-L1 protein and/or exosomal PD-L1 protein. Suitably, the treatment arises through adaptive immunity (e.g. cellular immunity and/or humoral immunity). According to another aspect the present invention provides a method of treating a proliferative disease, such as cancer by reducing the amount of extra-cellular PD-L1 protein and/or increasing the amount of OX40-L protein, produced by proliferative disease cells in a patient comprising administering a therapeutically effective amount of NUC-7738 to a patient in need thereof. Suitably, the extra-cellular PD-L1 is soluble PD-L1 protein and/or exosomal PD-L1 protein.

According to another aspect the present invention provides use of NUC-7738 in the manufacture of a medicament for use in a method of treating a proliferative disease, such as cancer by reducing the amount of extra-cellular PD-L1 protein and/or increasing the amount of OX40-L protein, produced by the diseased cells. Suitably, the extra-cellular PD-L1 is soluble PD-L1 protein and/or exosomal PD-L1 protein. Suitably, the proliferative disease is cancer. In a particular embodiment, reducing the amount of extra-cellular PD-L1 protein, such as soluble PD-L1 protein and/or exosomal PD-L1 protein, produced by the cancer cells renders the cancer cells more susceptible to targeting by the host immune system, such as the patient's cellular immune system.

According to one aspect the present invention provides NUC-7738 for use in the treatment of a proliferative disease, such as cancer, by reducing the amount of extra-cellular PD-L1 protein produced by the diseased cells. Suitably, the extra-cellular PD-L1 is soluble PD-L1 protein and/or exosomal PD-L1 protein. Suitably, the treatment arises through adaptive immunity (e.g. cellular immunity and/or humoral immunity).

According to another aspect the present invention provides a method of treating cancer by reducing the amount of extra-cellular PD-L1 protein, such as soluble PD-L1 protein and/or exosomal PD-L1 protein, produced by cancer cells in a patient comprising administering a therapeutically effective amount of NUC-7738 to a patient in need thereof.

According to another aspect the present invention provides use of NUC-7738 in the manufacture of a medicament for use in a method of treating cancer by reducing the amount of extra-cellular PD-L1 protein, such as soluble PD-L1 protein and/or exosomal PD-L1 protein, produced by the cancer cells. In a particular embodiment, reducing the amount of extra-cellular PD-L1 protein, such as soluble PD-L1 protein and/or exosomal PD-L1 protein, produced by the cancer cells renders the cancer cells more susceptible to targeting by the host immune system, such as the patient's cellular immune system. The ability of NUC-7738 to cause a reduction in the production and release of extra-cellular PD-L1 protein, such as soluble PD-L1 and exosomal PD-L1 protein, supports that NUC-7738 is an immune-sensitiser and can be used as an immune sensitising agent. This role is further supported by the finding that NUC-7738 also causes an increase in the expression and production of OX40-L by the cancer cells, which interacts with OX40 on the surface of T lymphocytes to increase their anti-neoplastic effectiveness.

According to another aspect the present invention provides NUC-7738 for use in the treatment of a proliferative disease by enhancing the patient's immune response against the proliferative disease. Suitably, the NUC-7738 enhances the patient's adaptive immune response. Suitably, NUC-7738 suppresses one or more immune blockers, such as extra cellular PD-L1 and/or OX40-L.

According to another aspect the present invention provides NUC-7738 for use as an immune-sensitiser. Suitably, the present invention provides NUC-7738 for use as an immune-sensitiser in the treatment of cancer.

According to another aspect the present invention provides the use of NUC-7738 in the manufacture of an immune-sensitiser medicament. Alternatively, the invention provides the use of NUC-7738 in the manufacture of a medicament for use in the treatment of cancer, wherein the NUC-7738 is an immune-sensitiser.

According to another aspect the present invention provides a method of potentiating the immune response to a tumour, the method comprising administering to a patient in need of such treatment a therapeutically effective amount of NUC-7738, alone or in combination with an immune oncology agent as described herein. In a particular embodiment, there is provided a method of potentiating the effect of an an immune oncology agent, the method comprising administering to a patient in need of such treatment a therapeutically effective amount of the immune oncology agent simultaneously or sequentially with NUC-7738.

According to another aspect the present invention provides NUC-7738 for use in the treatment of cancer by increasing the amount of OX40-L protein produced by the cancer cells. Suitably, the treatment of cancer arises through adaptive immunity (e.g. cellular immunity and/or humoral immunity).

According to another aspect the present invention provides a method of treating cancer by increasing the amount of OX40-L protein produced by the cancer cells in a patient comprising administering a therapeutically effective amount of NUC-7738 to a patient in need thereof.

According to another aspect the present invention provides use of NUC-7738 in the manufacture of a medicament for use in a method of treating cancer by increasing the amount of OX40-L protein produced by the cancer cells.

In a particular embodiment, increasing the amount of OX40-L protein produced by the cancer cells renders the cancer cells more susceptible to targeting by the host immune system, such as the patient's cellular immune system by promoting effector T cell activation.

According to another aspect the present invention provides NUC-7738 for use in the treatment of cancer by reducing a blockage against targeting the cancer cells by the patient's immune system. Suitably “reducing a blockage” comprises reducing suppression or a suppressor of the patient's immune system.

According to another aspect the present invention provides NUC-7738 for use in the treatment of cancer by enhancing the patient's immune response against the cancer. Suitably, the NUC-7738 augments the patient's immune system against the cancer.

According to another aspect the present invention provides use of NUC-7738 in the manufacture of a medicament for use in a method for treating cancer by enhancing the patient's immune response against the cancer.

According to another aspect the present invention provides a method of treating cancer by enhancing the patient's immune response against the cancer, wherein the method comprises administering to the patient in need thereof a therapeutically effective amount of NUC-7738.

According to another aspect the present invention provides a combination comprising NUC-7738 and an immune oncology agent.

In particular embodiments, the immune oncology agent is an immune checkpoint inhibitor, a cancer vaccine, antibody therapy, or an adoptive cell therapy such as CAR-T cell.

In particular embodiments, the immune oncology agent is an immune checkpoint inhibitor selected from pembrolizumab, cemiplimab, dostarlimab, nivolumab, durvalumab, ipilimumab, atezolizumab, and avelumab. Suitably, the immune checkpoint inhibitor is an anti-PD-1 antibody selected from pembrolizumab, cemiplimab, dostarlimab and nivolumab. In a particular embodiments, the immune checkpoint inhibitor is pembrolizumab.

According to another aspect the present invention provides a pharmaceutical product comprising NUC-7738 and an immune oncology agent.

In a particular embodiment, the immune oncology agent is an immune checkpoint inhibitor. The immune checkpoint inhibitor may be an antibody against an immune checkpoint protein.

Suitably the immune checkpoint inhibitor is an antibody selected from the group consisting of: an anti-PD-L1 antibody, an anti-PD-1 antibody, an anti-CTLA-4 antibody, an anti-LAG-3 antibody, an anti-TIGIT antibody, an anti-BTLA antibody, an anti-TIM-3 antibody, an anti-CD40 antibody and an anti-CD40L antibody.

Suitably the immune checkpoint inhibitor is selected from pembrolizumab, cemiplimab, dostarlimab, nivolumab, durvalumab, ipilimumab, atezolizumab, and avelumab. Suitably, the immune checkpoint inhibitor is an anti-PD-1 antibody selected from pembrolizumab, cemiplimab, dostarlimab and nivolumab. In a particular embodiment the immune checkpoint inhibitor is pembrolizumab.

In one embodiment, the pharmaceutical product may comprise a kit of parts comprising separate formulations of NUC-7738 and an immune checkpoint inhibitor. The separate formulations of NUC-7738 and the immune checkpoint inhibitor may be administered sequentially and/or simultaneously.

In another embodiment the pharmaceutical product is a kit of parts which comprises:
a first container comprising NUC-7738, such as NUC-7738 in association with a pharmaceutically acceptable adjuvant, diluent or carrier; and
a second container comprising an immune checkpoint inhibitor such as an immune checkpoint inhibitor in association with a pharmaceutically acceptable adjuvant, diluent or carrier, and
a container means for containing said first and second containers.

In one embodiment, the pharmaceutical product may comprise a one or more unit dosage forms (e.g. vials, tablets or capsules in a blister pack). In one embodiment, each unit dose comprises only one agent selected from NUC-7738 and the immune checkpoint inhibitor. In another embodiment, the unit dosage form comprises both the NUC-7738 compound and the immune checkpoint inhibitor.

According to another aspect the present invention provides a combination comprising NUC-7738 and an immune checkpoint inhibitor as defined herein for use in the treatment of a proliferative disease. Suitably, the proliferative disease is cancer. In particular embodiments, the proliferative disease is one that is normally treated with an immune checkpoint inhibitor, such as one selected from the group consisting of: pembrolizumab, cemiplimab, dostarlimab, nivolumab, durvalumab, ipilimumab, atezolizumab, and avelumab. Table 1 provides a list of diseases that particular checkpoint inhibitor agents can be used to treat.

The following combinations and diseases are specific embodiments:
NUC-7738 in combination with nivolumab for use in treating a cancer selected from: melanoma (including cutaneous melanoma), renal cell carcinoma, non-small cell lung cancer, mesothelioma, classical hodgkin lymphoma, squamous cell carcinoma, urothelial carcinoma, colorectal cancer, hepatocellular carcinoma, eosophogeal cancer, gastric cancer and, gastroesophageal junction cancer
NUC-7738 in combination with durvalumab for use in treating a cancer selected from: urothelial carcinoma, non-small cell lung-cancer (NSCLC) and extensive stage small cell lung-cancer.
NUC-7738 in combination with pembrolizumab for use in treating a cancer selected from: melanoma (including cutaneous melanoma), non-small cell lung cancer, urothelial carcinoma, classical Hodgkin lymphoma, head and neck squamous cell carcinoma, renal cell carcinoma, colorectal cancer and esophageal carcinoma.
NUC-7738 in combination with dostarlimab for use in treating a cancer selected from: mismatch repair deficient (dMMR) recurrent or advanced endometrial cancer.
NUC-7738 in combination with ipilimumab for use in treating a cancer selected from: melanoma (including cutaneous melanoma), renal cell carcinoma, mon-small cell lung cancer, mesothelioma, classical hodgkin lymphoma, colorectal cancer, hepatocellular carcinoma and eosophogeal cancer.
NUC-7738 in combination with cemiplimab for use in treating a cancer selected from: cutaneous squamous cell carcinoma, basal cell carcinoma and non-small cell lung cancer.
NUC-7738 in combination with atezolizumab for use in treating a cancer selected from: melanoma (including cutaneous melanoma), non-small cell lung cancer, small cell lung cancer, urothelial carcinoma, an hepatocellular carcinoma.
NUC-7738 in combination with avelumab for use in treating a cancer selected from: merkel cell carcinoma, urothelial carcinoma and renal cell carcinoma.

Suitably, the proliferative disease is cancer and the cancer cells express high levels of soluble or exosomal PD-L1. Suitably, the proliferative disease is cancer and the cancer cells express low levels of OX40L.

According to another aspect the present invention provides a method of treating a proliferative disease in a subject in need thereof comprising administering to said subject a combination comprising NUC-7738 and an immune checkpoint inhibitor as defined herein. In particular embodiments, the proliferative disease is one that is normally treated with an immune checkpoint inhibitor, such as one selected from the group consisting of: pembrolizumab, cemiplimab, dostarlimab, nivolumab, durvalumab, ipilimumab, atezolizumab, and avelumab. Table 1 provides a list of diseases that particular checkpoint inhibitor agents can be used to treat.

The following combinations and diseases are specific embodiments:
NUC-7738 in combination with nivolumab for use in treating a cancer selected from: melanoma (including cutaneous melanoma), renal cell carcinoma, non-small cell lung cancer, mesothelioma, classical hodgkin lymphoma, squamous cell carcinoma, urothelial carcinoma, colorectal cancer, hepatocellular carcinoma, eosophogeal cancer, gastric cancer and, gastroesophageal junction cancer
NUC-7738 in combination with durvalumab for use in treating a cancer selected from: urothelial carcinoma, non-small cell lung-cancer (NSCLC) and extensive stage small cell lung-cancer.
NUC-7738 in combination with pembrolizumab for use in treating a cancer selected from: melanoma (including cutaneous melanoma), non-small cell lung cancer, urothelial carcinoma, classical Hodgkin lymphoma, head and neck squamous cell carcinoma, renal cell carcinoma, colorectal cancer and esophageal carcinoma.
NUC-7738 in combination with dostarlimab for use in treating a cancer selected from: mismatch repair deficient (dMMR) recurrent or advanced endometrial cancer.
NUC-7738 in combination with ipilimumab for use in treating a cancer selected from: melanoma (including cutaneous melanoma), renal cell carcinoma, mon-small cell lung cancer, mesothelioma, classical hodgkin lymphoma, colorectal cancer, hepatocellular carcinoma and eosophogeal cancer.
NUC-7738 in combination with cemiplimab for use in treating a cancer selected from: cutaneous squamous cell carcinoma, basal cell carcinoma and non-small cell lung cancer.
NUC-7738 in combination with atezolizumab for use in treating a cancer selected from: melanoma (including cutaneous melanoma), non-small cell lung cancer, small cell lung cancer, urothelial carcinoma, an hepatocellular carcinoma.
NUC-7738 in combination with avelumab for use in treating a cancer selected from: merkel cell carcinoma, urothelial carcinoma and renal cell carcinoma.

Suitably, the immune checkpoint inhibitor is pembrolizumab, cemiplimab, dostarlimab or nivolumab.

Suitably, the proliferative disease is cancer and the cancer cells express high levels of soluble or exosomal PD-L1. Suitably, the proliferative disease is cancer and the cancer cells express low levels of OX40L.

According to another aspect the present invention provides the use of NUC-7738 in the manufacture of a medicament for treating of a proliferative disease in combination with an immune checkpoint inhibitor as defined herein.

In particular embodiments, the proliferative disease is one that is normally treated with an immune checkpoint inhibitor, such as one selected from the group consisting of: pembrolizumab, cemiplimab, dostarlimab, nivolumab, durvalumab, ipilimumab, atezolizumab, and avelumab. Table 1 provides a list of diseases that particular checkpoint inhibitor agents can be used to treat.

The following combinations and diseases are specific embodiments:
NUC-7738 in combination with nivolumab for use in treating a cancer selected from: melanoma (including cutaneous melanoma), renal cell carcinoma, non-small cell lung cancer, mesothelioma, classical hodgkin lymphoma, squamous cell carcinoma, urothelial carcinoma, colorectal cancer, hepatocellular carcinoma, eosophogeal cancer, gastric cancer and, gastroesophageal junction cancer
NUC-7738 in combination with durvalumab for use in treating a cancer selected from: urothelial carcinoma, non-small cell lung-cancer (NSCLC) and extensive stage small cell lung-cancer.
NUC-7738 in combination with pembrolizumab for use in treating a cancer selected melanoma (including cutaneous melanoma), non-small cell lung cancer, urothelial from: carcinoma, classical Hodgkin lymphoma, head and neck squamous cell carcinoma, renal cell carcinoma, colorectal cancer and esophageal carcinoma.
NUC-7738 in combination with dostarlimab for use in treating a cancer selected from: mismatch repair deficient (dMMR) recurrent or advanced endometrial cancer.
NUC-7738 in combination with ipilimumab for use in treating a cancer selected from: melanoma (including cutaneous melanoma), renal cell carcinoma, mon-small cell lung cancer, mesothelioma, classical hodgkin lymphoma, colorectal cancer, hepatocellular carcinoma and eosophogeal cancer.
NUC-7738 in combination with cemiplimab for use in treating a cancer selected from: cutaneous squamous cell carcinoma, basal cell carcinoma and non-small cell lung cancer.
NUC-7738 in combination with atezolizumab for use in treating a cancer selected from: melanoma (including cutaneous melanoma), non-small cell lung cancer, small cell lung cancer, urothelial carcinoma, an hepatocellular carcinoma.
NUC-7738 in combination with avelumab for use in treating a cancer selected from: merkel cell carcinoma, urothelial carcinoma and renal cell carcinoma.

Suitably, the proliferative disease is cancer and the cancer cells express high levels of soluble or exosomal PD-L1. Suitably, the proliferative disease is cancer and the cancer cells express low levels of OX40L. Suitably the immune checkpoint inhibitor is selected from pembrolizumab, cemiplimab, dostarlimab, nivolumab, durvalumab, ipilimumab, atezolizumab, and avelumab. Suitably, the immune checkpoint inhibitor is an anti-PD-1 antibody selected from pembrolizumab, cemiplimab, dostarlimab and nivolumab. A particularly suitable immune checkpoint inhibitor for use in combination with NUC-7738 for treating cancer, such as cutaneous melanoma, is pembrolizumab.

In particular embodiments of this aspect of the invention the NUC-7738 and immune checkpoint inhibitor are administered to the subject simultaneously or sequentially.

According to another aspect the present invention provides NUC-7738 as defined herein for use in the treatment of a proliferative disease, wherein the NUC-7738 is for simultaneous or sequential administeration with an immune checkpoint inhibitor as defined herein. Suitably, the immune checkpoint inhibitor is selected from the group consisting of: pembrolizumab, cemiplimab, dostarlimab, nivolumab, durvalumab, ipilimumab, atezolizumab, and avelumab. Suitably, the proliferative disease is one that is normally treated with an immune checkpoint inhibitor, such as pembrolizumab, cemiplimab, dostarlimab or nivolumab.

According to another aspect the present invention provides a method of treating a proliferative disease in a subject in need thereof comprising administering to said subject a combination comprising NUC-7738 and an immune checkpoint inhibitor as defined herein, wherein the NUC-7738 and immune checkpoint inhibitor are administered simultaneously or sequentially. Suitably, the immune checkpoint inhibitor is selected from the group consisting of: pembrolizumab, cemiplimab, dostarlimab, nivolumab, durvalumab, ipilimumab, atezolizumab, and avelumab. Suitably, the immune checkpoint inhibitor is an anti-PD-1 antibody selected from pembrolizumab, cemiplimab, dostarlimab and nivolumab. Suitably, the proliferative disease is one that is normally treated with an immune checkpoint inhibitor, such as pembrolizumab, cemiplimab, dostarlimab or nivolumab.

According to another aspect the present invention provides an immune checkpoint inhibitor as defined herein for use in the treatment of a proliferative disease, wherein the immune checkpoint inhibitor is for simultaneous or sequential administeration with NUC-7738 as defined herein. Suitably, the immune checkpoint inhibitor is selected from the group consisting of: pembrolizumab, cemiplimab, dostarlimab, nivolumab, durvalumab, ipilimumab, atezolizumab, and avelumab. Suitably, the immune checkpoint inhibitor is pembrolizumab, cemiplimab, dostarlimab or nivolumab. Suitably, the proliferative disease is one that is normally treated with an immune checkpoint inhibitor, such as pembrolizumab, cemiplimab, dostarlimab or nivolumab.

According to another aspect the present invention provides the use of an immune checkpoint inhibitor in the manufacture of a medicament for treating of a proliferative disease in combination with NUC-7738 as defined herein. Suitably, the immune checkpoint inhibitor is selected from the group consisting of: pembrolizumab, cemiplimab, dostarlimab, nivolumab, durvalumab, ipilimumab, atezolizumab, and avelumab. Suitably, the immune checkpoint inhibitor is pembrolizumab, cemiplimab, dostarlimab or nivolumab. Suitably, the proliferative disease is one that is normally treated with an immune checkpoint inhibitor, such as pembrolizumab, cemiplimab, dostarlimab or nivolumab.

In particular embodiments of this aspect of the invention the NUC-7738 and immune checkpoint inhibitor are administered to the subject simultaneously or sequentially.

Suitably the proliferative disease is cancer. In particular embodiments, the proliferative disease is one that is normally treated with an immune checkpoint inhibitor, such as pembrolizumab, cemiplimab, dostarlimab or nivolumab. According to another aspect the present invention provides NUC-7738 for use in the treatment of cancer in a subject whose cancer cells express high levels of soluble or exosomal PD-L1.

According to another aspect the present invention provides the use of NUC-7738 in the manufacture of a medicament for treating cancer in a subject whose cancer cells express high levels of extra-cellular PD-L1 protein, such as soluble or exosomal PD-L1 protein, wherein the treatment comprises administering the NUC-7738 in combination with an immune checkpoint inhibitor as defined herein. Optionally, prior to the treating the patient is tested to determine whether they have a cancer whose cancer cells express high levels of extra-cellular PD-L1 protein, such as soluble PD-L1 protein or exosomal PD-L1 protein. Suitably, the immune checkpoint inhibitor is pembrolizumab, cemiplimab, dostarlimab or nivolumab.

According to another aspect the present invention provides use of NUC-7738 in the manufacture of a medicament for use in a method of treating cancer by reducing the amount of extra-cellular PD-L1 protein, such as soluble protein and/or exosomal PD-L1 protein, produced by the cancer cells, wherein the method comprises determining whether a subject has a cancer whose cancer cells express high levels of extra-cellular PD-L1 protein, wherein if the subject has a cancer whose cancer cells express high levels of extra-cellular PD-L1 protein, such as soluble PD-L1 protein or exosomal PD-L1 protein the subject is administered a combination of NUC-7738 and an immune checkpoint inhibitor as defined herein. Suitably, the immune checkpoint inhibitor is pembrolizumab, cemiplimab, dostarlimab or nivolumab.

According to another aspect the present invention provides a method of treating cancer in a subject whose cancer cells express high levels of extra-cellular PD-L1 protein, such as soluble PD-L1 protein or exosomal PD-L1 protein, comprising administering to the subject a therapeutically effective amount of NUC-7738 alone or in combination with an immune checkpoint inhibitor. Suitably, the immune checkpoint inhibitor is pembrolizumab, cemiplimab, dostarlimab or nivolumab.

According to another aspect the present invention provides a method of treating cancer in a subject whose cancer cells express high levels of extra-cellular PD-L1, such as soluble PD-L1 protein or exosomal PD-L1 protein, comprising contacting the cancer cells with a therapeutically effective amount of NUC-7738. In particular embodiments of this aspect of the invention the cancer cells are within a subject or patient. Thus, suitably, the cancer cells are contacted with a therapeutically effective amount of NUC-7738 by administering to the subject a therapeutically effective amount of NUC-7738, alone or in combination with an immune checkpoint inhibitor. Suitably, the immune checkpoint inhibitor is pembrolizumab, cemiplimab, dostarlimab or nivolumab.

Any immune checkpoint inhibitor may be used in the combination therapy defined herein.

In particular embodiments, the checkpoint inhibitor is selected from the group consisting of: an anti-PD-L1 antibody, an anti-PD-1 antibody, an anti-CTLA-4 antibody, an anti-LAG-3 antibody, an anti-TIGIT antibody, an anti-BTLA antibody, an anti-TIM-3 antibody, an anti-CD40 antibody and an anti-CD40L antibody.

Suitably, the immune checkpoint inhibitor is an anti-PD-1 antibody selected from pembrolizumab, cemiplimab, dostarlimab or nivolumab.

In particular embodiments, the proliferative disease is cancer characterised by cancer cells that express high levels of extra-cellular PD-L1 protein, e.g. soluble PD-L1 or exosomal PD-L1. In particular embodiments, the proliferative disease is one that is normally treated with an immune checkpoint inhibitor, such as pembrolizumab, cemiplimab, dostarlimab or nivolumab. Table 1 provides a list of diseases that particular checkpoint inhibitor agents can be used to treat.

According to another aspect the present invention provides a method of determining whether a patient will benefit from treatment with NUC-7738, the method comprising: determining the level of soluble PD-L1 and/or exosomal PD-L1 in a biological sample from the patient; wherein if the level of soluble PD-L1 and/or exosomal PD-L1 produced by the cancer cells in the biological sample is elevated or reduced compared to a reference value the patient will benefit from treatment with NUC-7738.

Suitably, the biological sample is a blood sample or a fraction therefrom (e.g. plasma or serum).

The various aspects of the invention are based in part on the finding that NUC-7738 is able to reduce the amount of extra-cellular PD-L1 protein, including soluble PD-L1 protein and exosomal PD-L1 protein produced by cancer cells. PD-L1 is known to mask the cancer cell from the immune system and elevated soluble PD-L1 in particular is a biomarker of poor prognosis and resistance to immune checkpoint inhibitors. Accordingly, the ability of NUC-7738 to reduce the amount of extra-cellular PD-L1 protein, such as soluble PD-L1 protein and/or exosomal PD-L1 protein, produced by the cancer cells provides new clinical opportunities for treating and managing cancers in patients. For example, by removing or lessening the camouflage that the cancer cell has from the immune system, thus allowing the host immune system to recognise and attack, include kill, the cancer cell. The ability of NUC-7738 to reduce the levels of extra-cellular PD-L1 protein, such as soluble PD-L1 protein and/or exosomal PD-L1 protein, will serve to enhance the effectiveness of an immune oncology agent, including immune checkpoint inhibitors, which provides for the ability to treat a cancer patient with a combination of NUC-7738 and an immune oncology agent. When the immune oncology agent is an immune checkpoint inhibitor, as described herein, it may also facilitate the use of a lower dose of the checkpoint inhibitor thus lessening the toxic effect of such agent. Certain cancer patients also develop resistance to immune checkpoint inhibitors which is often associated with an increase in soluble PD-L1 levels. The ability of NUC-7738 to target and reduce the amount of soluble or exosomal PD-L1 is predicted to remove the resistance/block and thus allow the patient to be re-treated with the same or an alternate immune checkpoint inhibitor or to be treated with an immune checkpoint inhibitor that would not otherwise be effective without an NUC-7738-induced reduction in soluble PD-L1. NUC-7738 can therefore also be used to treat patients that are or have developed or have pre-existing resistance to an immune checkpoint inhibitor.

Thus, in particular embodiments of any of the aspects of the invention the patient or subject is one that is resistant to or has developed resistance to an immune checkpoint inhibitor. Such a person is one where established doses of the immune checkpoint inhibitor do not provide recognised clinical signals of efficacy. Suitably a subject/patient that has developed resistance to an immune checkpoint inhibitor will have previously shown clinical signals of efficacy, such as maintenance or reduction in cancer mass or reduction in markers of cancer, progression free survival, but the effectiveness of the immune checkpoint inhibitor has then reduced or ceased. This is an indication that the cancer has become resistant to the agent. Suitably a subject/patient that is resistant to (has pre-existing resistance to) an immune checkpoint inhibitor will have markers, such as low mutational burden, microsatellite stable, low tumour infiltrating lymphocytes, high levels of immunosuppressive cytokines, PTEN deletion, indicating pre-existing resistance to immune checkpoint inhibitor therapy (see Liu et al., Am J Clin Dermatol. 20 (1): 41-54, 2019).

The various aspects of the invention are also based upon the finding that NUC-7738 is able to increase the amount of OX40L produced by cancer cells. Stimulating OX40 has shown it to be a candidate for therapeutic immunization strategies for cancer.

OX40 signalling is important for priming and generation of CD4 and CD8 T cell immunity, and in development of T cell memory. OX40 is expressed on T-cells following recognition of foreign or aberrant peptides by T-cell receptors on their cell surface. Binding of OX40 by its ligand OX40L enhances the activation of T-cells and has been shown to augment antitumour immune responses (Weinberg et al. I J Immunol. 164 (4): 2160-2169, 2000). The OX40/0X40L axis is therefore recognised as an attractive targets for therapy of autoimmune and inflammatory disease, as well as for the design of vaccination and therapeutic adjuvant strategies for infectious disease and cancer (see e.g.: Deng et al. “OX40 (CD14) and OX40 ligand, important immune checkpoints in cancer”. Onco Targets Ther. 12:7347-7353, 2019; Roszik et al. “TNFSF4 (OX40L) expression and survival in locally advanced and metastatic melanoma”. Cancer Immunology, Immunotherapy. 68:1493-1500, 2019; Dannull J, Nair S, Su Z, Boczkowski D, DeBeck C, Yang B. “Enhancing the immunostimulatory function of dendritic cells by transfection with mRNA encoding OX40 ligand. Blood. 105:3206-3213, 2005).

Reference to NUC-7738 in any aspect or embodiment of the invention includes NUC-7738 as the(S)-phosphate diastereoisomer or as the (R)-phosphate diastereoisomer or as a mixture of phosphate diastereoisomers; it also includes the compound when in the form of a free base or it may be in the form of a pharmaceutically acceptable salt; and, it also includes a pharmaceutical composition comprising the NUC-7738.

Reference to an immune checkpoint inhibitor in any aspect or embodiment of the invention includes the agent or a pharmaceutically acceptable salt thereof; it also includes a pharmaceutical composition comprising the immune checkpoint inhibitor.

The ability of NUC-7738 to reduce the amount/levels of soluble PD-L1 protein and/or exosomal PD-L1 protein and/or increase the amount/level of OX40L protein provides new therapeutic opportunities for treating cancer.

### DESCRIPTION OF THE DRAWINGS

FIG. 1A and FIG. 1B. Show the concentration of soluble PD-L1 in melanoma (A375) and lung cancer (A549) cell lines, before and after treatment with NUC-7738, by ELISA.

FIG. 2A and FIG. 2B. Show the soluble PD-L1 transcript fold change in melanoma (A375) and lung cancer (A549) cell lines, before and after treatment with NUC-7738.

FIG. 3A, FIG. 3B, FIG. 3C, and FIG. 3D. Show exosomal PD-L1 from four patients, before and after treatment with NUC-7738. Pre-Cycle 1 (PreC1, untreated), 24 hr Post-Cycle 1 (24 hr) and Pre-Cycle 2 (PreC2, 15 days after initial cycle). Cycle in this case is each time a patient receives the drug.

FIG. 4A and FIG. 4B. Show OX40L mRNA expression levels in melanoma (A375) and lung cancer (A549) cell lines treated with NUC-7738.

### DETAILED DESCRIPTION OF THE INVENTION

Further features and embodiments of the above defined aspects are described herein below in headed sections. Each section is combinable with any of the above-mentioned aspects in any compatible combination.

The following definitions may be useful in the understanding of the invention.

As used herein, “treatment” refers to clinical intervention in an attempt to alter the natural course of the subject or cell being treated and can be performed before or during the course of clinical pathology. Desirable effects of treatment include preventing the occurrence or recurrence of a disease or a disorder or symptom thereof, alleviating a disorder or symptom of the disease, diminishing any direct or indirect pathological consequences of the disease, decreasing the rate of disease progression, ameliorating or palliating the disease state, and achieving remission or improved prognosis. In some embodiments, methods and compositions of the disclosure are useful in attempts to delay development of a disease or disorder. The treatment is applicable to both human therapy and veterinary applications. The terms disease and disorder as used herein are generally interchangeable unless the context dictates otherwise.

“Amelioration” includes that the diseases or disorders (or symptoms associated therewith) described herein are alleviated, diminished, decreased and/or palliated.

Soluble PD-L1 is secreted from tumour cells and is present in plasma as a free soluble protein. Exosomal PD-L1 refers to PD-L1 protein bound to exosome membrane which are released as extra-cellular vesicles from cells. As used herein, extra-cellular PD-L1 refers to PD-L1 protein which is released/shed from the cell and is not cell bound. PD-L1 protein released from the cell may be circulatory PD-L1. Soluble PD-L1 and exosomal PD-L1 are examples of extra-cellular PD-L1.

Exosomes, a type of extra-cellular vesicles (EV), are small, membrane-enclosed structures likely shed from the surface of healthy or damaged cells under conditions such as cell activation, growth, and apoptosis. These vesicular structures contain substantial amounts of biologically active proteins, lipids, and nucleic acids acquired from their parental cells. EVs are commonly found in blood (e.g. plasma), urine, saliva, tears, and many other body fluids. EVs and/or biomarkers present in/on or associated therewith have been proposed for use in the diagnosis, prognosis, and monitoring of disease and health conditions.

Extra-cellular vesicles can be isolated and purified using polymer-based resin available as a kit ExoQuick® ULTRA EV from Systems Bioscience. The isolated EV can then be quantified for their protein content using, for example, the Bicinchoninic Acid (BCA) protein quantification assay. Suitably, quantification of exosomes protein marker CD81 may be used. In addition, particle size distribution of EV may be determined using NanoSight.

Engagement of PD-1 by the PD-L1 ligand can alter the tumour microenvironment and supress an endogenous antitumor immune response mediated by CD8+T-cells and other components of the cellular immune system. Kim et al. (Experimental & Molecular Medicine 51:94, 2019) demonstrated that exosomes derived from non-small cell lung cancer cells that express PD-L1 play a role in immune escape by reducing T-cell activity and promoting tumour growth. They found that the abundance in exosomes expressing PD-L1 isolated from the plasma of patients correlated with PD-L1 positivity in tumour tissues. They found that exosomes can impair immune functions by reducing cytokine production and inducing apoptosis in CD8+T cells which indicate that tumour-derived exosomes expressing PD-L1 may be an important mediator of tumour immune escape.

Soluble PD-L1 (sPD-L1) is a predictive and prognostic biomarker for cancer patients selected for or receiving immune checkpoint blockade treatment (see Oh, S. Y., Kim, S., Keam, B. et al. Soluble PD-L1 is a predictive and prognostic biomarker in advanced cancer patients who receive immune checkpoint blockade treatment. Sci Rep 11, 19712 (2021). https://doi.org/10.1038/s41598-021-99311-y). Similarly, exosomal PD-L1 (xPD-L1) can also be used as a biomarker for cancer patients selected for or receiving immune checkpoint blockade treatment. (Chen et al. “Exosomal PD-L1 contributes to immunosuppression and is associated with anti-PD-1 response”. Nature. 560:382-386, 2018; Li et al. J of Translational Medicine. “Clinical significance of PD-L1 expression in serum-derived exosomes in NSCLC patients”. 17, 225, 2019). As used herein, the terms “exosomal PD-L1”, “xPD-L1”, “extra-vesicle PD-L1”, and “EV PD-L1” are used interchangeably.

The term “high levels of soluble PD-L1 or exosomal PD-L1” refers to an increase over the normal level, typically above a clinically relevant reference level. Thus, high levels of soluble PD-L1 refers to an amount that is outside the usual/normal amount. Such deviation from the normal level may be an increase of, for example, 10%, 20%, 30%, 40%, 50%, 60%, 70%, 80%, 90%, 100%, 125%, 150%, 200% or more.

The usual or normal level can be determined from measurements of the level from a population of subjects that are considered to be healthy, or unaffected by the disease mediated by or associated with high levels of soluble PD-L1 and applying standard statistical measures. Such value can then be used to decide on the appropriate “reference level” against which to determine whether the level of soluble PD-L1 or exosomal PD-L1 produced by a cell or population of cells is normal, high, or low.

Low level OX40L expression is associated with poor prognosis and grade (see Roszik et al. supra). Accordingly, OX40L may be used as a biomarker to select suitable patients for treatment with an immune checkpoint inhibitor and/or the methods of the present invention. For example, tumors with low OX40L being targets for therapies that increase tumoral OX40L expression.

The term “low levels of OX40L” refers to a decrease from the normal level, typically below a clinically relevant reference level. Thus, low levels of OX40L refers to an amount that is outside the usual/normal amount. Such deviation from the normal level may be a decrease of, for example, 10%, 20%, 30%, 40%, 50%, 60%, 70%, 80%, 90%, 100%, 125%, 150%, 200% or more.

A “clinically relevant reference level” is a value that has been determined from appropriate clinical studies as an amount/level (e.g. threshold) of, e.g. soluble PD-L1 or exosomal PD-L1 or OX40L level, for classifying whether the amount is high or not. It is a value that can be used for making clinical decisions. The use of such reference values are well known for diagnostic tests and for making clinical decisions.

The term “administered” or “administering” in all of its grammatical forms means administration of a therapeutically effective dose of the NUC-7738 as the sole therapeutic agent or in combination with another therapeutic agent, such as an immune checkpoint inhibitor as described herein, to a subject/patient. It is envisaged that the NUC-7738 is suitably in the form of a composition, preferably pharmaceutical composition, that may be employed in co-therapy approaches, i.e. in co-administration with other medicaments or drugs, for example, other medicaments for treating a disease as described herein and/or any other therapeutic agent which might be beneficial in the context of the methods of the present disclosure.

An “effective amount”, as used herein, refers to an amount effective, at dosages and for periods of time necessary to achieve the desired therapeutic or prophylactic result. The exact dose will depend on the purpose of the treatment and will be ascertainable by one skilled in the art using known techniques.

By “therapeutically effective amount” it is meant the amount of an agent (e.g. compound such as NUC-7738) that produces the effects for which it is administered. A “therapeutically effective amount” of NUC-7738 as described herein may vary according to factors such as age, body weight, general health, sex, diet, time of administration, drug interaction and the severity of the disorder may be necessary, and will be ascertainable with routine experimentation by those skilled in the art. Sometimes, the term “therapeutically effective amount” may sometimes be interchangeably used herein with the term “pharmaceutically effective amount”. The “therapeutically effective amount” of NUC-7738 when used in combination with a particular immune checkpoint inhibitor may be different to the “therapeutically effective amount” when NUC-7738 is administered alone (e.g. as monotherapy) and it may be different to the “therapeutically effective amount” when used in combination with a different immune checkpoint inhibitor.

A therapeutically effective amount, when used in the present application, is also one in which any toxic or detrimental effects of the NUC-7738 are outweighed by the therapeutically beneficial effects.

Cancer drugs are usually administered in treatment cycles. The term “treatment cycle” refers to a period of treatment that is repeated on a regular schedule. Any treatment cycle may include a period of rest (no treatment) prior to receiving the next dose of treatment. For example, treatment given for three weeks followed by one week of rest is one treatment cycle of a 28 day cycle. A treatment cycle is normally between 2 and 6 weeks. Multiple treatment cycle can follow one after the other in series so that when you get to the end of the cycle, it starts again with the next cycle. A series of cycles is called a course of treatment. A course of treatment may take between 3 to 6 months but it can be more or less than that or it may be indefinite. During that time, the patient may receive between 3 to more than 10 cycles of treatment.

The drug may be administered on each day of a cycle or on particular days of a cycle as part of a treatment regimen which reflects the days or times when the drug is administered, or not. Thus, for example, NUC-7738 may be administered in a 21-day treatment cycle, with for example drug administered on days 1, 8 and 15 which would mean the patient should receive NUC-7738 once every week because 7 days after the last dose in cycle 1 (day 15) would be day 1 of cycle 2.

In some embodiments, a “subject”, when used in the present application, is a vertebrate. In certain particular embodiments, the vertebrate is a mammal. Mammals include, but are not limited to, primates (including human and non-human primates) and rodents (e.g., mice and rats). In certain particular embodiments, a mammal subject is a human. In some further embodiments, a human subject, synonymous with an individual, is a particularly preferred subject. As used herein, the term “subject” is used synonymously with the term “patient”. Suitably, the subject(s)/patient(s) are human(s).

References herein to NUC-7738 being administered “in combination with” an immunotherapy agent, such as an checkpoint inhibitor (e.g. a CTLA4, LAG3, PD-1 or PD-L1 inhibitor), or vice versa, unless otherwise stated otherwise, include the agents being administered sequentially or simultaneously with one another.

As used herein “simultaneous administration” refers to therapy in which the both agents (e.g. NUC-7738 and immune oncology agent) are administered at substantially the same time. An example of administration simultaneously is where the two agents are mixed together in the same infusion bag or administered at the same time using a “Y-line” from different infusion bags. An example of simultaneous administration is where the duration of administration of the two agents overlaps at least to some extent.

As used herein “sequential administration” means that one agent is administered after the other (i.e. separately), however, the time period between the administration of each agent is such that both agents are capable of acting therapeutically concurrently. Thus, administration “sequentially” may permit one agent to be administered within seconds, minutes, or a matter of hours after the other provided the circulatory half-life of the first administered agent is such that they are both concurrently present in therapeutically effective amounts. The time delay between the administration of the agents may vary depending on the exact nature of the agents, the interaction therebetween, and their respective half-lives. Suitably, the two agents are administered within 5, 10, 15, 20, 30, 60, 120 minutes of each other. The two agents need not be administered on the same day, indeed they could be administered as part of a treatment regime involving a cycle of administration (e.g. agent 1 given on days 1, 8 and 15 of a 21 day cycle and agent 2 given on days 3, 10 and 17 of the 21 day cycle; such cycle of treatment could then be repeated one or more times. By way of another example, agents 1 and 2 could be given on days 1 of a 21 day cycle an only agent 2 given on one or more other days of the 21 day cycle). Suitably, the two agents are administered within 1, 2, 3, 4, 5, 6, 7, 8, 9, 10, 11, 12, 13 or 14 days of each other. In a particular embodiment of any aspect the present invention when NUC-7738 and an immune checkpoint inhibitor are administered sequentially, the NUC-7738 is administered first. In a particular embodiment of any aspect of the present invention when NUC-7738 and an immune checkpoint inhibitor are administered sequentially, the immune checkpoint inhibitor is administered first. “sequential administration” requires that the two agents are administered in the same treatment cycle or as part of a course of treatment.

As used herein, a “pharmaceutical product” refers to a product comprising a pharmaceutical. For instance, examples of a pharmaceutical product include a medical device, a pharmaceutical composition and a kit of parts suitably comprising one or more devices, containers and/or pharmaceuticals.

As used herein, the term “immune-sensitiser” refers to an agent that serves to activate or enhance the host immune system. In the context of NUC-7738 it can do this by reducing the amount of soluble or exosomal PD-L1 protein present or by increasing the amount of OX40L protein that is expressed on cancer cells which can bind to OX40 on T-lymphocytes and other components of the cellular immune system resulting in enhanced T-cell activation. Thus, the immune sensitiser effectively switches on the immune system or removes a brake (suppressor) that impedes efficient activation of the immune system, so that the immune system becomes sensitized to the cancer. As described herein PD-L1 is the immunosuppressive PD-1 ligand, thus by reducing PD-L1 the immune environment is more conducive to cancer cell death mediated by the cellular immune system.

As used herein, an “immune oncology agent” refers to any agent which is capable of eliciting a host immune response. Such agents are typically used in immunotherapy which is a type of cancer treatment that helps the subject's immune system fight cancer. Examples of immune oncology agents include immune checkpoint inhibitors in particular an anti-PD-1 antibody, an anti-PD-L1 antibody, an anti-LAG3 antibody, and anti-CTLA-4 antibody, an anti-TIM3 antibody, and anti-BTLA antibody, and anti-OX40 antibody or an anti-OX40L antibody], oncology vaccines and adoptive cell therapy such as CAR-T cells (CAR-T therapy). In particular embodiment, the immune oncology agent is an immune checkpoint inhibitor, as described herein (e.g. see section entitled “Immune checkpoint inhibitors”) or an adoptive cell therapy such as CAR-T cell population or a cancer vaccine, as described herein and known to the person of skill in the art. As used herein, the term “immunotherapy agent” is synonymous with “immune oncology agent”.

### Adoptive Cell Therapies

Adoptive cell therapy, also known as cellular immunotherapy, is a form of treatment that uses the cells of the host's (patient's) immune system to attack cancer cells.

Certain of these approaches involve directly isolating immune cells from a patient and expanding their numbers, whereas others involve genetically engineering the isolated immune cells (via gene therapy) to enhance their ability to identify and attack cancer cells.

In particular embodiments, the adoptive cell therapy is selected from: CAR-T, TIL and NK cell therapies.

### Tumor-Infiltrating Lymphocyte (TIL) Therapy

Most cancer patients have different types of naturally occurring T cells that are capable of targeting the cancer cells. In particular, “killer” T cells are capable of recognizing and eliminating cancer cells directly. However, such cells must be activated before they can effectively kill cancer cells, and then they must be able to maintain that activity for a sufficiently long time to sustain an effective anti-tumor response. Furthermore, in order to mount an effective attack sufficient numbers of these T cells must be produced.

One form of adoptive cell therapy that attempts to address these issues is called tumor-infiltrating lymphocyte (TIL) therapy. This approach harvests naturally occurring T cells that have already infiltrated a patients' tumor, and then activates and expands them. Next, large numbers of these activated T cells are re-infused into the patient, where they can then seek out and destroy tumors.

Granhøj et al. review the advances in TIL adoptive cell therapy, including the principles and techniques used (Expert Opin Biol Ther. 22 (5): 627-641, 2020).

### Engineered T-Cell Receptor (TCR) Therapy

Certain patients may not have T cells that have already recognized their tumors, or if they do, these T cells may not be capable of being activated and expanded to sufficient numbers to enable rejection of their tumors. For these patients, an approach known as engineered T cell receptor (TCR) therapy may be appropriate.

This approach also involves taking T cells from the patient, but rather than simply activating and expanding the available anti-tumor T cells, the T cells are altered to present a new T cell receptor that enables them to target specific cancer antigens.

### CAR-T Cell Therapy

The previously mentioned TIL and TCR therapies can only target and eliminate cancer cells that present their antigens in a certain context (when the antigens are bound by the major histocompatibility complex, or MHC).

Approaches designed to address this limitation involve engineering the T cell with a synthetic receptor known as a CAR, which stands for chimeric antigen receptor.

CARs are synthetic molecules comprising an ectodomain that functions as a high affinity ligand (most often derived from an antibody and manufactured as a single chain variable fragment-scFv) specific for a target cell surface antigen and an endodomain that ensures forceful activation and proliferation of the modified T cells in an HLA-independent manner. The basic configuration of the CAR endodomain comprises one or two co-stimulatory molecule domains (derived from CD28, 41-BB or OX-40) placed in tandem with the CD3z domain.

A key advantage of CARs is their ability to bind to cancer cells even if their antigens aren't presented on the surface via MHC, which can render more cancer cells vulnerable to their attacks. However, CAR-T cells can only recognize antigens that themselves are naturally expressed on the cell surface, so the range of potential antigen targets is smaller than with TCRs.

CAR-T cells (chimeric antigen receptor T-cells) are T-cells, typically isolated from the same patient to be treated (autologous) but could be from a different donor source (allogeneic), which are engineered to express proteins on their surface, called chimeric antigen receptors (CARs). The CARs recognise and bind to specific antigens on the surface of cancer cells and thus target the T-cell to the cancer cell. The engineered CAR-T cells are first expanded in the laboratory and then infused back into the patient where the CAR-T cells can then multiply, target to the cancer cells and kill them.

Since 2017, six CAR-T cell therapies have been approved by the US Food and Drug Administration (FDA). All are approved for the treatment of blood cancers, including lymphomas, some forms of leukemia and, most recently multiple myeloma.

The effectiveness of CAR-T cell therapy on solid tumours is constrained by the inhibitory impacts of immune checkpoints in the microenvironment of solid tumours (Ma, S., Li, X., Wang, X., Cheng, L., Li, Z., Zhang, C., et al. Current progress in CAR-T cell therapy for solid tumors. Int. J. Biol. Sci. 15, 2548-2560, 2019; Shi, X., Zhang, D., Li, F., Zhang, Z., Wang, S., Xuan, Y., et al. Targeting glycosylation of PD-1 to enhance CAR-T cell cytotoxicity. J. Hematol. Oncol. 12:127, 2019).

Programmed cell death protein-1 (PD-1)-mediated immunosuppression has been proposed to contribute to the limited clinical efficacy of CAR-T cell therapy in solid tumours. PD-1-mediated immunosuppression has been proposed to be involved in CAR-T cell hypofunction (Moon EK, Wang LC, Dolfi DV, Wilson CB, Ranganathan R, Sun J, et al. Multifactorial T-cell hypofunction that is reversible can limit the efficacy of chimeric antigen receptor-transduced human T cells in solid tumors. Clin Cancer Res. 2014; 20:4262-73.) and the combination of CAR-T cell therapy with anti-PD-1 antibody treatment has shown encouraging antitumor activity in patients Chong EA, Melenhorst JJ, Lacey SF, Ambrose DE, Gonzalez V, Levine BL, et al. PD-1 blockade modulates chimeric antigen receptor (CAR)-modified T cells: refueling the CAR. Blood. 129:1039-41, 2017; and Adusumilli PS, Zauderer MG, Rusch VW, O'Cearbhaill R, Zhu A, Ngai D, et al. Regional delivery of mesothelin-targeted CAR T cells for pleural cancers: safety and preliminary efficacy in combination with anti-PD-1 agent. J Clin Oncol. 37:2511, 2019).

It is believed that lessening the inhibitory effects of immune checkpoints (like PD-1/PD-L1) would augment CAR-T cell therapy. The ability of NUC-7738 to reduce the amount of soluble and exosomal PD-L1 is believed to reduce the inhibitory effects of immune checkpoints and so augment CAR-T cell therapy.

Additionally, the ability of NUC-7738 to increase expression of OX40L is likely to be beneficial for CAR-T therapy. For example, Zhang, Huihui, et al. (“A chimeric antigen receptor with antigen-independent OX40 signaling mediates potent antitumor activity.” Science translational medicine 13.578 (2021): eaba7308.) demonstrate that CAR-T and co-stimulation of OX-40 in tumour cells mediates potent antitumour activity. Thus, it is believed that the ability of NUC-7738 to increase the amount of OX40L will reduce the inhibitory effects of immune checkpoints and so augment CAR-T cell therapy.

### Natural Killer (NK) Cell Therapy

Adoptive cell therapy strategies have begun to incorporate other immune cells, such as Natural Killer (NK) cells. One application being explored in the clinic involves equipping these NK cells with cancer-targeting CARs.

Biederstädt and Rezvani (Int J Hematol. 114 (5): 554-571, 2021) provides an overview on current trends and evolving concepts to genetically engineer the next generation of CAR-NK therapies

In particular embodiments, there is provided NUC-7738 for use in combination with an adoptive cell therapy. In a particular embodiment, there is provided NUC-7738 for use in augmenting the effects of an adoptive cell therapy. Suitably, the adoptive cell therapy is selected from CAR-T, NK and TIL.

### Cancer Vaccines

Cancer vaccines involve the administration of a therapy that results in the presentation of cancer antigens to the immune system, boosting the immune system's ability to find cancer cells that express these antigens and eliminate them. Cancer vaccines can be viral, RNA, DNA, or peptide-based and specific to proteins that are on expressed on particular cancer cells (e.g. Sipuleucel-T for prostate cancer or Talimogene laherparepvec for advanced melanoma skin cancer).

Preclinical studies combining cancer vaccines with immune checkpoint blockade have been promising. For example, van Elsas, et al. (van Elsas A, Hurwitz AA, Allison JP. Combination immunotherapy of B16 melanoma using anti-cytotoxic T lymphocyte-associated antigen 4 (CTLA-4) and granulocyte/macrophage colony-stimulating factor (GM-CSF)-producing vaccines induces rejection of subcutaneous and metastatic tumors accompanied by autoimmune depigmentation. J Exp Med. 190:355-66, 1999) examined the effectiveness of cytotoxic T lymphocyte-associated antigen 4 (CTLA-4) blockade, alone or in combination with a granulocyte/macrophage colony-stimulating factor (GM-CSF) expressing tumor cell vaccine, on rejection of the highly tumorigenic, poorly immunogenic murine melanoma B16-BL6 and found that tumours could be eradicated in 80% (68/85) of the cases using combination treatment, whereas each treatment by itself showed little or no effect.

It is believed that lessening the inhibitory effects of immune checkpoints (like PD-1/PD-L.1) would augment cancer vaccine therapy. The ability of NUC-7738 to reduce the amount of soluble and exosomal PD-L1 is believed to reduce the inhibitory effects of immune checkpoints and so augment cancer vaccine therapy. Furthermore, stimulation of OX40 on immune cells has been proposed as a strategy to enhance the effectiveness of vaccines (Panagioti et al. Front Immunol. 20 (8): 144, 2017) therefore NUC-7738's ability to increase OX40L expression may enhance the effectiveness of cancer vaccines.

In a particular embodiment, there is provides NUC-7738 for use in combination with a cancer vaccine. In a particular embodiment, there is provided NUC-7738 for use in augmenting the effects of a cancer vaccine.

### “NUC-7738”

The present invention relates to medical uses of NUC-7738, alone or in combination with an immune oncology agent, in particular medical uses for the treatment of proliferative diseases such as cancer, by reducing the level of extra-cellular PD-L1 protein produced by the cancer cell(s) or increasing the level of OX40-L produced by the cancer cell(s). Suitably, the extra-cellular PD-L1 protein is soluble PD-L1 or exosomal PD-L1. The reduction in level of soluble PD-L1 or exosomal PD-L1 is believed to arise due to NUC-7738 reducing the translation of soluble/exosomal PD-L1. The increase in level of OX40-L is believed to arise due to NUC-7738 enhancing the transcription of OX40-L.

The compound 3′-deoxyadenosine-5′-O-[phenyl(benzyloxy-L-alaninyl)] phosphate (also referred to as NUC-7738), is a phosphoramidate derivative of 3′-deoxyadenosine. The NUC-7738 compound, including its synthesis, is disclosed in WO2016/083830 (Nucana). Phamaceutical compositions comprising phosphoramidate molecules, including NUC-7738, are disclosed in WO2017/109491 (Nucana). WO2018229493 (Nucana) and WO2018229495 (Nucana) disclose particular synthesis routes for NUC-7738.

NUC-7738 has the structure shown in Formula 1.

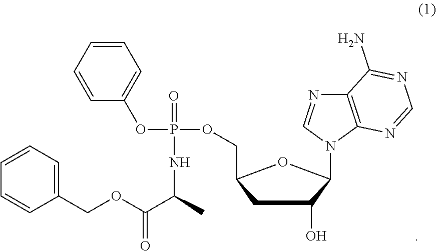

NUC-7738 comprises a chiral centre at the phosphorous atom. The NUC-7738 may be present as a mixture of phosphate diastereoisomers, as the(S)-epimer at the phosphorus atom in substantially diastereomerically pure form or as the (R)-epimer at the phosphorus atom in substantially diastereomerically pure form. ‘Substantially diastereomerically pure’ is defined for the purposes of this invention as a diastereomeric purity of greater than about 90%. If present as a substantially diastereoisomerically pure form, the NUC-7738 may have a diastereoisomeric purity of greater than 95%, 98%, 99%, or even 99.5%. Alternatively, the NUC-7738 may be present as a mixture of phosphate diastereoisomers.

The (R)- and/or(S)-phosphate diastereoisomers of the NUC-7738 can be obtained in substantially diastereomerically pure form by chromatography, e.g. HPLC optionally using a chiral column. Alternatively, the (R)- and/or(S)-phosphate diastereoisomers of NUC-7738 can be obtained in substantially diastereomerically pure form by crystallisation from an appropriate solvent or solvent system. In a further alternative, the (R)- and/or(S)-phosphate diastereoisomers of NUC-7738 can be synthesised as a diastereomerically pure form using a diastereoselective synthesis. It may be that any combination of these techniques could be used to provide a diastereomerically pure form, e.g. a diastereoselective synthesis followed by crystallisation or chromatography. It may be more convenient to make a protected form of the NUC-7738 diastereoisomeric mixture, to separate the protected forms of the NUC-7738 diastereoisomers (e.g. using chromatography or crystallisation) and to subsequently remove the protecting groups to provide the substantially diastereoisomerically pure NUC-7738.

The NUC-7738 as employed herein may be in the form of a free base or it may be in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate and hemicalcium salts.

The NUC-7738 may exist in a single crystal form or in a mixture of crystal forms or it may be amorphous. Thus, the NUC-7738 compound intended for medical use according to the invention may be administered as crystalline or amorphous products. It may be obtained, for example, as solid plugs, powders, or films by methods such as precipitation, crystallization, freeze drying, or spray drying, or evaporative drying. Microwave or radio frequency drying may be used for this purpose.

Unless the context dictates otherwise, reference to NUC-7738 in any aspect or embodiment of the invention includes NUC-7738 as the(S)-phosphate diastereoisomer or as the (R)-phosphate diastereoisomer or as a mixture of phosphate diastereoisomers; it also includes the compound when in the form of a free base or it may be in the form of a pharmaceutically acceptable salt; and, it also includes a pharmaceutical composition comprising the NUC-7738 (e.g. NUC-7738 formulations).

“NUC-7738” formulations

NUC-7738, including a pharmaceutically acceptable salt thereof, may be used alone but will generally be administered in the form of a pharmaceutical composition in which NUC-7738 is in association with one or more pharmaceutically acceptable excipients, such as an adjuvant, diluent or carrier. Pharmaceutical excipients are substances other than the pharmacologically active drug or prodrug which are included in the manufacturing process or are contained in a finished pharmaceutical product dosage form. Conventional procedures for the selection and preparation of suitable pharmaceutical formulations are described in, for example, “Pharmaceuticals—The Science of Dosage Form Designs”, M. E. Aulton, Churchill Livingstone, 1988.

Suitably, any reference herein to the use of “NUC-7738” may also refer to the use of a pharmaceutical composition comprising NUC-7738.

Depending on the mode of administration of NUC-7738, the pharmaceutical composition which is used to administer NUC-7738, including any pharmaceutically acceptable salt thereof, will preferably comprise from 0.05 to 99% w (percent by weight) NUC-7738, or a pharmaceutically acceptable salt thereof, more preferably from 0.05 to 80% w NUC-7738, or a pharmaceutically acceptable salt thereof, still more preferably from 0.10 to 70% w NUC-7738, and even more preferably from 0.10 to 50% w NUC-7738, all percentages by weight being based on total composition.

NUC-7738, may be administered orally. For oral administration NUC-7738 may be admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

In particular embodiments, the NUC-7738 or a composition comprising NUC-7738 is administered parenterally, and in particular, intravenously. Parenteral application methods include, for example, intracutaneous, subcutaneous, intramuscular, intratracheal, intranasal, intravitreal or intravenous injection and infusion techniques, e.g. in the form of injection solutions, infusion solutions or tinctures. For parenteral (e.g. intravenous) administration NUC-7738 may be administered as a sterile aqueous or oily solution. Aqueous formulations for intravenous administration, particularly those of the free base of NUC-7738 may also contain a pharmaceutically acceptable polar organic solvent, e.g. dimethylacetamide, and one or more solubilisers or other additives as excipients.

NUC-7738 or formulations comprising NUC-7738 according to or for use in the present invention may be used in the treatment of a human or other animal, for example to treat commercial animals such as livestock or companion animals such as cats, dogs, etc. Suitably, NUC-7738 or formulations comprising NUC-7738 according to or for use in the present invention are for use or can be used in the treatment of the human body.

NUC-7738 according to or for use in the present invention may be obtained, stored and/or administered in the form of a pharmaceutically acceptable salt. Suitable pharmaceutically acceptable salts include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulfate, hemioxalate and hemicalcium salts. Preferably, the NUC-7738 compound is not in the form of a salt, i.e. it is in the form of the free base/free acid.

When administered to a subject/patient, the dosage administered will, of course, vary depending on the precise mode of administration, the treatment desired and the disease/disorder indicated. Dosage levels, dose frequency, and treatment durations are also expected to differ depending on the formulation and clinical indication, age, and co-morbid medical disorders of the patient. The size of the dose for therapeutic purposes of the NUC-7738 or a pharmaceutical formulation comprising it may also vary according to the nature and severity of the disorders, the age and sex of the animal or patient and the route of administration, according to well-known principles of medicine.

A pharmaceutical formulation typically takes the form of a composition in which active compounds, or pharmaceutically acceptable salts thereof, are in association with one or more pharmaceutically acceptable excipients. One such pharmaceutically acceptable excipient in the formulations of the invention is the polar aprotic solvent. Conventional procedures for the selection and preparation of suitable pharmaceutical formulations are described in, for example, “Pharmaceuticals—The Science of Dosage Form Designs”, M. E. Aulton, Churchill Livingstone, 1988.

The formulations may be suitable for topical application (e.g. to the skin), for oral administration or for parenteral (e.g. intravenous administration).

Any solvents used in pharmaceutical formulations of the invention should be pharmaceutical grade, by which it is meant that they have an impurity profile which renders them suitable for administration (e.g. intravenous administration) to humans.

For oral administration the formulations of the invention may comprise the active compound admixed with an adjuvant or a carrier, for example, lactose, saccharose, sorbitol, mannitol; a starch, for example, potato starch, corn starch or amylopectin; a cellulose derivative; a binder, for example, gelatine or polyvinylpyrrolidone; and/or a lubricant, for example, magnesium stearate, calcium stearate, polyethylene glycol, a wax, paraffin, and the like, and then compressed into tablets. If coated tablets are required, the cores, prepared as described above, may be coated with a concentrated sugar solution which may contain, for example, gum arabic, gelatine, talcum and titanium dioxide. Alternatively, the tablet may be coated with a suitable polymer dissolved in a readily volatile organic solvent.

For the preparation of soft gelatine capsules, the active compounds may be admixed with, for example, a vegetable oil or polyethylene glycol. Hard gelatine capsules may contain granules of the compound using either the above-mentioned excipients for tablets. Also, liquid or semisolid formulations of the active compounds may be filled into hard gelatine capsules.

Liquid preparations for oral application may be in the form of syrups or suspensions, for example, solutions containing the compound of the invention, the balance being sugar and a mixture of ethanol, water, glycerol and propylene glycol. Optionally such liquid preparations may contain colouring agents, flavouring agents, sweetening agents (such as saccharine), preservative agents and/or carboxymethylcellulose as a thickening agent or other excipients known to those skilled in art.

Preferably, however the formulation comprising NUC-7738 according to or for use in the invention is for parenteral (e.g. intravenous) administration or for dilution to form a formulation for parenteral (e.g. intravenous) administration. For parenteral (e.g. intravenous) administration the NUC-7738 compound may be administered as a sterile aqueous or oily solution. Preferably, the active NUC-7738 compound is administered as a sterile aqueous solution.

The pharmaceutical composition comprising NUC-7738 according to or for use in the methods of the invention will preferably comprise from 0.05 to 99% w (percent by weight) NUC-7738, more preferably from 0.05 to 80% w NUC-7738, still more preferably from 0.10 to 70% w NUC-7738, and even more preferably from 0.10 to 50% w NUC-7738, all percentages by weight being based on total composition.

Suitably the NUC-7738 may be administered in a pharmaceutically effective or acceptable amount for the in vivo treatment of a patient. Suitably the patient is a human, though the patient could be another animal.

Suitably, the NUC-7738 including pharmaceutical composition thereof may be administered to a human or other animal in accordance with the aforementioned methods of treatment/medical uses in an amount sufficient to produce a therapeutic effect.

Reference to NUC-7738 in the context of a pharmaceutical composition, e.g., a pharmaceutical composition comprising NUC-7738, includes NUC-7738 as the(S)-phosphate diastereoisomer or as the (R)-phosphate diastereoisomer or as a mixture of phosphate diastereoisomers; it also includes the compound when in the form of a free base or it may be in the form of a pharmaceutically acceptable salt.

### Therapeutically Effective Doses of NUC-7738

A therapeutically effective amount of NUC-7738 may be an amount sufficient to induce death of cancer cells.

There are various different ways in which the amount of a therapeutically effective compound, such as NUC-7738, to be administered to a patient may be calculated and expressed. One such way which is considered particularly relevant in doses of agents for the prevention or treatment of cancer, is in the amount of the agent to be administered per unit of body surface area of the patient. Such doses are typically expressed in terms of the amount of the agent (which may be determined by mass) per square meter (m²) of surface area.

Uses of NUC-7738 for the treatment of cancer may utilise a weekly dose of between 300 mg/m² and 1600 mg/m². Such treatments may, for example utilise a weekly dose of between 500 mg/m² and 1150 mg/m², or a weekly dose between 900 mg/m² and 1350 mg/m². The nominal dose may be selected from: 900 mg/m², 1100 mg/m², 1125 mg/m² and 1350 mg/m². Uses of NUC-7738 for the treatment of cancer may utilise less frequent dosing, e.g. Q2W, Q3W or Q4W, with the dosing adjusted accordingly as appropriate.

A chosen weekly dose of NUC-7738 for use according to the present invention may be provided in a single incidence of administration, or in multiple incidences of administration during a week. For example, a weekly dose of a compound of the invention may be provided in two incidences of administration, in three incidences of administration, or more. Thus, in the case of a weekly dose of 900 mg/m², this may be achieved by three administrations of 300 mg/m² over the course of a week, or two administrations of 450 mg/m² during a week. Similarly, in the case of a weekly dose of 1350 mg/m², this may be achieved by three administrations of 450 mg/m² over the course of a week, or two administrations of 675 mg/m² over the course of a week. If dosing is Q2W, Q3W or Q4W the dose administered can be determined based on the chosen weekly dose.

A suitable amount of NUC-7738 for use according to the present invention to be administered in a single incidence of treatment in order to provide a required dose of this compound over the course of week may be between approximately 900 mg/m² and 1350 mg/m².

The weekly dose of NUC-7738 for use according to the present invention may decrease over the course of treatment. For example, treatment may be started at a weekly dose of around 1350 mg/m², 1125 mg/m², 1100 mg/m², 900 mg/m², or 750 mg/m², and over the course of treatment the dose needed may decrease to around 750 mg/m² (in cases where the initial dose is above this amount), to around 625 mg/m², or 500 mg/m² or even around 375 mg/m².

Doses of NUC-7738 for use according to the present invention can, of course, be presented in other manners. The most common of these is the amount of the active agent to be provided per unit body mass. It has been calculated that for an average human patient a dose of 1 mg/m² is equivalent to approximately 0.025 mg/kg body mass. Accordingly, the data indicate that a compound of the invention is effective for the treatment of relapsed or refractory cancer at doses ranging from approximately 6.25 mg/kg to approximately 25 mg/kg. A suitable dose may, for example, be of between about 9.5 mg/kg and 22.5 mg/kg. In a suitable embodiment a compound of the invention achieves effective treatment of relapsed or refractory cancers when patients are provided with weekly doses ranging between approximately 12.5 mg/kg and 20.5 mg/kg.

The NUC-7738 may be administered in a series of treatment cycles, with each cycle being of any duration of time, for example, 1, 2, 3, 4, 5, 6 weeks long, or 14, 15, 16, 17, 18, 19, 20, 21, 22, 23, 24, 25, 26, 27, 28, 35, or 42 days long, or longer. In particular embodiments, the NUC-7738 is administered to a patient in a series of 21-day treatment cycles, such series being 2, 3, 4, 5, 6, 7 8, 9, 10 or more treatment cycles. In other particular embodiments, the NUC-7738 is administered to a patient in a series of 42-day treatment cycles, such series being 2, 3, 4, 5, 6, or more treatment cycles.

It may be that the NUC-7738 is administered once, twice or thrice in a 21 day treatment cycle. In one embodiment, in each treatment cycle the NUC-7738 is administered on day 1 of a 21 day treatment cycle. In another embodiment, in each treatment cycle the NUC-7738 is administered on day 1 and day 8 of a 21 day treatment cycle. In another embodiment, in each treatment cycle the NUC-7738 is administered on day 1, 8 and 15 of a 21 day treatment cycle.

It may be that the NUC-7738 is administered 1-6 times in a 42 day treatment cycle. In one embodiment, in each treatment cycle the NUC-7738 is administered on day 1 of a 42 day treatment cycle. In another embodiment, in each treatment cycle the NUC-7738 is administered on day 1 and day 8 of a 42 day treatment cycle. In another embodiment, in each treatment cycle the NUC-7738 is administered on day 1, 8 and 15 of a 42 day treatment cycle. In another embodiment, in each treatment cycle the NUC-7738 is administered on day 1, 8, 15 and 22 of a 42 day treatment cycle. In another embodiment, in each treatment cycle the NUC-7738 is administered on day 1, 8, 15, 22 and 29 of a 42 day treatment cycle. In another embodiment, in each treatment cycle the NUC-7738 is administered on day 1, 8, 15, 22, 29 and 35 of a 42 day treatment cycle.

Considerations regarding formulations of NUC-7738 suitable for use in the methods of treatment and medical uses of the present invention are described elsewhere in this disclosure. In the case of injectable formulations of a compound of the invention, these may be administered intravenously. Intravenous administration may be achieved over any suitable time frame, for example in a ten minute injection, or the like.

### Therapeutically Effective Doses of an Immune Oncology Agent, Such as an Immune Checkpoint Inhibitor

A therapeutically effective amount of an immune oncology agent can be determined by a person skilled in the art using conventional and existing knowledge of the dosages approved by the health authority for such agents as monotherapy, or standard clinical studies. A therapeutically effective amount of a checkpoint inhibitor may be an amount sufficient to induce death of cancer cells.

By way of example, the following table (Table 1) is a non-exhaustive list of approved immune checkpoint inhibitors that may be suitable for use in the methods of treatment and medical uses of the present invention. In certain embodiments, one or more of the diseases listed in Table 1 is treated in any of the various aspects of the present invention as set out herein.

| Agent | Disease | dose |
|---|---|---|
| nivolumab | Melanoma (including cutaneous melanoma), | 240 mg every 2 weeks |
| | renal cell carcinoma, Non-small cell lung | or 480 mg every |
| | cancer, mesothelioma, classical hodgkin | 4 weeks |
| | lymphoma, squamous cell carcinoma, | |
| | urothelial carcinoma, colorectal cancer, | |
| | hepatocellular carcinoma, eosophogeal cancer, | |
| | gastric cancer and, gastroesophageal junction | |
| | cancer | |
| durvalumab | Urothelial carcinoma,, non-small cell lung- | 10 mg/kg every |
| | cancer (NSCLC) and extensive stage small cell | 2 weeks; 1500 mg every |
| | lung-cancer | 3 weeks or 1500 mg |
| | | every 4 weeks |
| pembrolizumab | Melanoma (including cutaneous melanoma), | 200 mg every 3 weeks |
| | Non-small cell lung cancer, | or 400 mg every 6 |
| | Urothelial carcinoma, | weeks |
| | Classical Hodgkin lymphoma, Head and neck | |
| | squamous cell carcinoma, | |
| | Renal cell carcinoma, | |
| | Colorectal cancer, | |
| | Oesophageal carcinoma | |
| dostarlimab | Mismatch repair deficient (dMMR) recurrent | 500 mg every 3 weeks |
| | or advanced endometrial cancer | or 1000 mg every 6 |
| | | weeks |
| ipilimumab | Melanoma, renal cell carcinoma, Non-small | 3 mg/kg every 3 weeks |
| | cell lung cancer, mesothelioma, classical | for 4 doses or 1 mg/kg |
| | hodgkin lymphoma, colorectal cancer, | every 6 weeks |
| | hepatocellular carcinoma and eosophogeal | |
| | cancer | |
| cemiplimab | Cutaneous squamous cell carcinoma, basal cell | 350 mg every 3 weeks |
| | carcinoma and non-small cell lung cancer | |
| atezolizumab | Melanoma, non-small cell lung cancer, small | 840 mg every 2 weeks, |
| | cell lung cancer, urothelial carcinoma, | 1200 mg every 3 |
| | hepatocellular carcinoma, | weeks, or 1680 mg |
| | | every 4 weeks |
| avelumab | Merkel cell carcinoma, urothelial carcinoma | 800 mg every 2 weeks |
| | and renal cell carcinoma | |

Appropriate doses of cancer vaccines and CAR-T cell compositions can also be determined by the person skilled in the art using conventional and existing knowledge of the dosages approved by the health authority for such agents as monotherapy or from standard clinical studies.

Examples of approved CAR-T therapies, the disease to be treated and the dose are included in the table below (Table 2):

| Agent | Disease | dose |
|---|---|---|
| idecabtagene | Multiple | 300 to 460 × 10⁶ CAR-positive T cells |
| vicleucel | Myeloma | |
| (Abecma) | | |
| lisocabtagene | DLBCL | 50 to 110 × 10⁶ CAR-positive viable T cells |
| maraleucel | | |
| (Breyanzi) | | |
| tisagenlecleucel | ALL, DLBCL | Pediatric and Young Adult B-cell ALL (up to 25 years |
| (Kymriah) | | of age) |
| | | 0.2 to 5.0 × 10⁶ CAR-positive viable T cells per kg of |
| | | body weight for patients 50 kg or less, or 0.1 to 2.5 × |
| | | 10⁸ CAR- positive viable T cells for patients more |
| | | than 50 kg |
| | | Adult Relapsed or Refractory Diffuse Large B-cell |
| | | Lymphoma |
| | | A single dose of KYMRIAH contains 0.6 to 6.0 × 10⁸ |
| | | CAR-positive viable T cells |
| brexucabtagene | ALL, MCL | MCL: 2 × 10⁶ CAR-positive viable Tcells per kg of |
| autoleucel | | body weight |
| (Tecartus) | | ALL: 1 × 10⁶ CAR-positive viable Tcells per kg of |
| | | body weight |
| axicabtagene | DLBCL | 2 × 10⁶ CAR-positive viable T cells per kg body |
| ciloleucel | | weight |
| (Yescarta) | | |
| ciltacabtagene | Multiple | 0.5-1.0 × 10⁶ CAR-positive viable T cells per kg body |
| autoleucel | Myeloma | weight in one infusion bag. |
| (Carvykti) | | |

### “NUC-7738 Medical Uses”

According to one aspect the present invention provides a method for treating a proliferative disease such as cancer. Suitably the method comprises administering a therapeutically effective amount of a composition to a subject in need of such composition, wherein said composition comprises NUC-7738. Suitably, the therapeutically effective amount is an amount capable of reducing the production of extra-cellular PD-L1 protein, such as soluble PD-L1 protein and/or exosomal PD-L1, by the cancer cells and/or increasing the production of OX40-L by the cancer cells. In particular embodiments, the proliferative disease is one in which high levels of soluble PD-L1 and/or exosomal PD-L1 contribute to the pathogenesis of the disease. E.g. cancer.

According to one aspect the present invention provides NUC-7738 for use in the treatment of cancer by reducing the amount of extra-cellular PD-L1 protein produced by the cancer cells. Suitably, the extra-cellular PD-L1 protein is soluble PD-L1 protein and/or exosomal PD-L1 protein. Suitably, the treatment of cancer arises through humoral immunity.

According to another aspect the present invention provides a method of treating cancer by reducing the amount of extra-cellular PD-L1 protein, such as soluble PD-L1 protein and/or exosomal PD-L1 protein, produced by the cancer cells in a patient comprising administering a therapeutically effective amount of NUC-7738 to a patient in need thereof.

According to another aspect the present invention provides use of NUC-7738 in the manufacture of a medicament for use in a method of treating cancer by reducing the amount of extra-cellular PD-L1 protein, such as soluble PD-L1 protein and/or exosomal PD-L1 protein, produced by the cancer cells.

According to another aspect the present invention provides NUC-7738 for use in the treatment of cancer by increasing the amount of OX40-L protein produced by the cancer cells.

According to another aspect the present invention provides a method of treating cancer by increasing the amount of OX40-L protein produced by the cancer cells in a patient comprising administering a therapeutically effective amount of NUC-7738 to a patient in need thereof.

According to another aspect the present invention provides use of NUC-7738 in the manufacture of a medicament for use in a method of treating cancer by increasing the amount of OX40-L protein produced by the cancer cells.

In a particular embodiment, increasing the amount of OX40-L protein produced by the cancer cells renders the cancer cells more susceptible to targeting by the host immune system, such as the patient's humoral immune system.

The ability of NUC-7738 to cause a reduction in the production and release of soluble PD-L1 and exosomal PD-L1 supports that NUC-7738 is an immune-sensitiser and can be used as an immune sensitising agent. This role is further supported by the finding that NUC-7738 also causes an increase in the expression and production of OX-40L by the cancer cells.

Thus, according to another aspect the present invention provides NUC-7738 for use as an immune-sensitiser. Suitably, the present invention provides NUC-7738 for use as an immune-sensitiser in the treatment of cancer.

According to another aspect the present invention provides the use of NUC-7738 in the manufacture of a medicament as an immune-sensitiser. Alternatively, the invention provides the use of NUC-7738 in the manufacture of a medicament for use in the treatment of cancer, wherein the NUC-7738 is an immune-sensitiser.

According to another aspect the present invention provides a method of potentiating the immune response to a tumour, the method comprising administering to a patient in need of such treatment a therapeutically effective amount of NUC-7738, alone or in combination with an immune oncology agent as described herein.

According to another aspect the present invention provides NUC-7738 for use in the treatment of cancer by reducing a blockage against targeting the cancer cells by the patient's immune system. Suitably reducing the blockage is removal of suppression or a suppressor of the patient's immune system. As described herein the suppressor could be extra-cellular PD-L1 protein, such as soluble PD-L1 protein and/or exosomal PD-L1 protein.

According to another aspect the present invention provides NUC-7738 for use in the treatment of cancer by enhancing the patient's immune response against the cancer.

According to another aspect the present invention provides use of NUC-7738 in the manufacture of a medicament for use in a method for treating cancer by enhancing the patient's immune response against the cancer.

According to another aspect the present invention provides a method of treating cancer by enhancing the patient's immune response against the cancer, wherein the method comprises administering to the patient in need thereof a therapeutically effective amount of NUC-7738.

In a particular embodiment of any of these aspects of the invention, administration of NUC-7738 to a patient enhances the patient's immune response against the proliferative disease.

In a particular embodiment of any of these aspects of the invention, administration of NUC-7738 to a patient reduces T-cell exhaustion.

In a particular embodiment of any of these aspects of the invention, administration of NUC-7738 counters the effects of extra-cellular PD-L1 protein.

In a particular embodiment of any of these aspects of the invention, administration of NUC-7738 counters the effects of soluble PD-L1 protein or exosomal PD-L1 protein.

In a particular embodiment of any of these aspects of the invention, the treatment of cancer arises through humoral immunity.

In particular embodiments of any of these aspects of the invention, the NUC-7738 is capable of reducing the levels of soluble PD-L1 produced by cancer cells.

In particular embodiments of any of these aspects of the invention, the NUC-7738 is capable of reducing the levels of exosomal PD-L1 produced by cancer cells.

In particular embodiments of any of these aspects of the invention, the NUC-7738 is capable of increasing the production of OX40-L by the cancer cells.

In particular embodiments of any of these aspects of the invention, the treatment with NUC-7738 causes a reduction in the level of extra-cellular PD-L1 protein.

In particular embodiments of any of these aspects of the invention, the treatment with NUC-7738 causes a reduction in the level of soluble PD-L1 protein or exosomal PD-L1 protein.

In particular embodiments, the NUC-7738 reduces the amount of extra-cellular PD-L1 produced by an affected cell (e.g. cancer cell) by at least 30%, such as at least 50%, at least 70%, at least 80% or at least 90% relative to the level prior to treatment.

In particular embodiments, the NUC-7738 reduces the amount of soluble PD-L1 protein and/or exosomal PD-L1 protein produced by an affected cell (e.g. cancer cell) by at least 30%, such as at least 50%, at least 70%, at least 80% or at least 90% relative to the level prior to treatment.

In particular embodiments of any of these aspects of the invention, the treatment with NUC-7738 causes an increase in the level of OX40-L protein produced by the proliferative (e.g. cancer) cells.

In particular embodiments of any of these aspects of the invention, the NUC-7738 is acting as an immune sensitiser. In particular embodiments, the NUC-7738 can be used to potentiate the effects of immunotherapy. In particular embodiments, the NUC-7738 can be used to potentiate the effects of an immune oncology agent. Suitably, the immune checkpoint inhibitor is an anti-PD-L1 or anti-PD-1 antibody. Suitably, the immune checkpoint inhibitor is selected from pembrolizumab, cemiplimab, dostarlimab, nivolumab, durvalumab, ipilimumab, atezolizumab, and avelumab. Suitably, the immune checkpoint inhibitor is an anti-PD-1 antibody selected from pembrolizumab, cemiplimab, dostarlimab and nivolumab. In a partcular embodiment for use in any of the aspects of the invention disclosed herein the immune checkpoint inhibitor is pembrolizumab.

Suitably the immunotherapy or immune oncology agent is an immune checkpoint inhibitor or a cancer vaccine or an adoptive cell therapy such as CAR-T cell therapy, as described herein.

As descibed further herein, in particular embodiments the treatment or method of treating may comprise administration of NUC-7738 in combination with an immunotherapy agent, such as an immune checkpoint inhibitor, an adoptive cell therapy such as a CAR-T cell, or a cancer vaccine, as described herein.

In particular embodiments, high levels of extra-cellular PD-L1 protein inhibits or blocks the host immune system from attacking the cancer cell.

In particular embodiments, high levels of extra-cellular PD-L1 protein inhibits or blocks the effect of an immune checkpoint inhibitor given to the patient.

In particular embodiments, high levels of soluble PD-L1 and/or exosomal PD-L1 inhibits or blocks the host immune system from attacking the cancer cell.

In particular embodiments, high levels of soluble PD-L1 and/or exosomal PD-L1 inhibits or blocks the effect of an immune checkpoint inhibitor given to the patient.

Accordingly, in particular embodiments, NUC-7738 is for use in treating a patient whose cancer cells express high levels of extra-cellular PD-L1 protein, such as soluble PD-L1 and/or exosomal PD-L1 protein.

In particular embodiments, the NUC-7738 reduces the expression of soluble PD-L1 and/or exosomal PD-L1 protein. In particular embodiments, the NUC-7738 reduces the transcription of the mRNA encoding soluble PD-L1 and/or exosomal PD-L1 protein.

In particular embodiments, low levels of OX40-L protein inhibits or blocks the host immune system from attacking the cancer cell.

In particular embodiments, low levels of OX40-L protein inhibits or blocks the effect of an immune checkpoint inhibitor given to the patient.

In particular embodiments, the NUC-7738 increases the expression of OX40-L protein by the cancer cells. In particular embodiments, the NUC-7738 increases the transcription of the mRNA encoding OX40-L.

In particular embodiments, the patient to be treated comprises a cancer whose cancer cells express high levels of soluble PD-L1 and/or exosomal PD-L1 protein.

In particular embodiments, the patient to be treated comprises a cancer whose cancer cells express low levels of OX40-L protein.

In particular embodiments, the patient has previously received treatment with an immune checkpoint inhibitor, optionally wherein said treatment has been stopped. Suitably, the previous treatment with an immune checkpoint inhibitor was stopped due to toxicity, relapse or the cancer becoming resistant to the previous treatment. Suitably, such patient can treated with NUC-7738 and the or an immune checkpoint inhibitor, but wherein the immune checkpoint inhibitor is administered at a lower dose that the approved monotherapy dose.

In particular embodiments, the patient to be treated has cancer that has become resistant to treatment with an immune checkpoint inhibitor.

In particular embodiments of any of these “NUC-7738 medical uses”, the NUC-7738 may be administered in one or more treatment cycles, such as 1, 2, 3, 4, 5, 6, 7, 8 or more treatment cycles.

### “Nuc-7738 Combinations”

Administration of NUC-7738 can reduce the amount of immune blockade by the cancer cells and so NUC-7738 can be used in combination with one or more immune oncology agents, such as an immune checkpoint inhibitor, an antibody therapy, a cancer vaccine (such as a therapeutic cancer vaccine), or an adoptive cell therapy such as CAR-T.

According to another aspect the present invention provides a method of potentiating the immune response to a tumour, the method comprising administering to a patient in need of such treatment a therapeutically effective amount of NUC-7738, alone or in combination with an immune oncology agent as described herein.

According to another aspect the present invention provides a method of potentiating the effect of an an immune oncology agent, the method comprising administering to a patient in need of such treatment a therapeutically effective amount of the immune oncology agent simultaneously or sequentially with NUC-7738.

According to another aspect the present invention provides a combination comprising NUC-7738 and an immune oncology agent.

In particular embodiments, the immune oncology agent is an immune checkpoint inhibitor, a cancer vaccine or an adoptive cell therapy such as CAR-T, as described herein.

In a particular embodiment, the immune oncology agent is an immune checkpoint inhibitor, as described herein. Suitably, the immune checkpoint inhibitor is selected from pembrolizumab, cemiplimab, dostarlimab, nivolumab, durvalumab, ipilimumab, atezolizumab, and avelumab.

According to another aspect the present invention provides a combination comprising NUC-7738 and an immune checkpoint inhibitor as defined herein for use in the treatment of a proliferative disease. Suitably, the proliferative disease is cancer, as described herein.

Because of the potentiating effect that NUC-7738 has on an immune checkpoint inhibitor it is predicted that the immune checkpoint inhibitor can be administered at a lower dose than the standard/approved monotherapy dose for the agent. Suitably, the dose of the immune checkpoint inhibitor can be 10%, 20%, 30%, 40%, 50%, 60%, 70%, or 80% of the approved monotherapy dose for the agent.

In particular embodiments, the immune checkpoint inhibitor for use in combination with the NUC-7738 is administered in a dose that is 75% or less than the standard or approved monotherapy dose for the agent. In particular embodiments, the immune checkpoint inhibitor for use in combination with the NUC-7738 is administered in a dose that is 50% or less than the standard or approved monotherapy dose for the agent. The approved dose refers to the dose approved/authorised by the appropriate health authority for a country or region, such as US Food and Drug Administration (FDA) for US, the European Medicines Agency (EMA) for Europe, and the Medicines and Healthcare Products Regulatory Agency (MHRA) for the UK, for use as a monotherapy agent in the relevant disease. For example, the anti-PD-1 antibody nivolumab is approved by MHRA for monotherapy treatment of adult patients with melanoma at a dose of 240 mg every 2 weeks or 480 mg every 4 weeks by intravenous infusion. A dose that was 50% of the approved monotherapy dose would therefore be 120 mg every 2 weeks or 240 mg every 4 weeks by intravenous infusion.

As described herein, and appreciated in the art, certain patients develop resistance to an administered immune checkpoint inhibitor. Other patients suffer from significant toxic side-effects and/or infusion-related reactions which may require dose delay or discontinuation. The ability of NUC-7738 to potentiate the effects of an immune checkpoint inhibitor should allow use of a lower dose of immune checkpoint inhibitor, which would allow patients to re-commence treatment that had been stopped due to resistance or re-commence treatment but at a lower dose for patients that had discontinued the original treatment due to toxic ide-effects or infusion-related reactions.

### “Combination Uses”

According to another aspect the present invention provides NUC-7738 for use in the combination treatment of a proliferative disease, wherein the NUC-7738 is administered in combination with an immune oncology agent as described herein. Suitably the immune oncology agent is an immune checkpoint inhibitor as defined herein. Suitably, the immune checkpoint inhibitor is selected from pembrolizumab, cemiplimab, dostarlimab, nivolumab, durvalumab, ipilimumab, atezolizumab, and avelumab. Suitably the immune checkpoint inhibitor is selected from pembrolizumab, cemiplimab, dostarlimab or nivolumab. Suitably the immun checkpointinhibitor is selected from nivolumab and pembrolizumab. Suitably, the immune checkpoint inhibitor is pembrolizumab. Suitably the proliferative disease is melanoma. Suitably the melanoma is cutaneous melanoma. Suitably the subject with melanoma, e.g. cutaneous melanoma, has received and optionally progressed on at least one prior therapy for the disease. Suitably the subject with melanoma, e.g. cutaneous melanoma, has received and optionally progressed on at least one prior immunotherapy treatment for the disease. Suitably the immune oncology agent is an adoptive cell therapy. Suitably the adoptive cell therapy is CAR-T cell therapy.

Thus, in a particular aspect the present invention provides NUC-7738 for use in the combination treatment of cancer, wherein the NUC-7738 is administered in combination with an immune checkpoint inhibitor selected from pembrolizumab, cemiplimab, dostarlimab, nivolumab, durvalumab, ipilimumab, atezolizumab, and avelumab. In particular embodiments, the NUC-7738 is administered in combination with nivolumab, cemiplimab, dostarlimab and/or pembrolizumab. In particular embodiments, the NUC-7738 is administered in combination nivolumab and/or pembrolizumab. In a particular embodiment, the NUC-7738 is administered in combination with pembrolizumab. In a particular embodiment the cancer is a solid tumour. In a particular embodiment the cancer is melanoma. In a particular embodiment the cancer is cutaneous melanoma. In a particular embodiment the cancer is advanced cutaneous melanoma. In a particular embodiment the cancer patient has advanced (including metastatic) cutaneous melanoma and has received at least one, such as 2, 3 or more, prior treatments for the cutaneous melanoma, optionally wherein the disease has progressed following said prior treatments. In such circumstances the combination treatment (e.g. NUC-7738+nivolumab and/or pembrolizumab) is second line, third line or further line treatment. In a particular embodiment the cancer patient has advanced (including metastatic) cutaneous melanoma and has already been treated with immunotherapy. In a particular embodiment, the patient with cancer, e.g. cutaneous melanoma, has been treated with an immune checkpoint inhibitor, such as one selected from pembrolizumab, cemiplimab, dostarlimab, nivolumab, durvalumab, ipilimumab, atezolizumab, and avelumab.

Thus in a particular aspect the present invention provides NUC-7738 for use in the combination treatment of melanoma, such as cutaneous melanoma, wherein the NUC-7738 is administered in combination with an immune checkpoint inhibitor selected from pembrolizumab, cemiplimab, dostarlimab, nivolumab, durvalumab, ipilimumab, atezolizumab, and avelumab. In particular embodiments, the NUC-7738 is administered in combination with nivolumab and/or cemiplimab and/or dostarlimab and/or pembrolizumab. In a particular embodiment, the NUC-7738 is administered in combination with pembrolizumab. In a particular embodiment, the NUC-7738 is administered in combination with pembrolizumab to a patient with cutaneous melanoma. In a particular embodiment, the NUC-7738 is administered in combination with pembrolizumab to a patient with advanced cutaneous melanoma. In a particular embodiment, the NUC-7738 is administered in combination with pembrolizumab to a patient with advanced cutaneous melanoma who has received at least one prior round of therapy, optionally who has received prior immunotherapy.

According to another aspect the present invention provides a method of treating a proliferative disease in a subject in need thereof comprising administering to said subject a combination comprising NUC-7738 and an an immune oncology agent, such as an immune checkpoint inhibitor as defined herein. Suitably, the proliferative disease is cancer. Suitably, the cancer selected from the group consisting of: melanoma (including cutaneous melanoma), lung cancer (including NSCLC), breast cancer, colorectal cancer, renal cancer, liver cancer, thyroid cancer, gastric cancer, pancreatic cancer, head and neck cancer, prostate cancer, bladder cancer, lymphoma, ovarian cancer, cervical cancer and endometrial cancer. In particular embodiments, the proliferative disease is one that is normally treated with an immune checkpoint inhibitor, such as pembrolizumab. Table 1 provides a list of diseases that particular checkpoint inhibitor agents can be used to treat. Suitably, the proliferative disease is cancer and the patient or tumour and its microenvironment exhibits high levels of soluble or exosomal PD-L1. Suitably, the proliferative disease is cancer and the cancer cells express low levels of OX40L.

According to another aspect the present invention provides the use of NUC-7738 in the manufacture of a medicament for treating a proliferative disease in combination with an an immune oncology agent, such as an immune checkpoint inhibitor as defined herein. Suitably, the proliferative disease is cancer.

In particular embodiments of these aspects of the invention the NUC-7738 and immune oncology agent, such as an immune checkpoint inhibitor are administered to the subject simultaneously or sequentially.

In particular embodiments of these aspects of the invention the NUC-7738 and immune oncology agent, such as an immune checkpoint inhibitor are administered to the subject in one or more treatment cycles, such as one or more 21 day or 42 day treatment cycles. The two agents can be administered on the same or different days of each treatment cycle. For example, when combining NUC-7738 and pembrolizumab, the pembrolizumab could be administered on day 1 of a 21 day treatment cycle and the NUC-7738 on days 1, 8 and 15 of the 21-day cycle; in another example, the pembrolizumab could be administered on day 1 of a 42 day treatment cycle and the NUC-7738 on days 1, 8, 15, 22, 29 and 35 of the 42-day cycle. In particular embodiments, the treatment cycle is repeated such that the patient may receive 2, 3, 4, 5, 6, 7, 8, 9, 10, 11, 12 or more treatment cycles for the course of treatment. When NUC-7738 is combined with an immune checkpoint inhibitor, such as pembrolizumab, on days when both agents are administered to the patient, the NUC-7738 can be administered first or second. Historically, it is usual for antibodies to be given before cytotoxic agents and so in one preferred embodiment the immune checkpoint inhibitor antibody, such as pembrolizumab, is administered before the NUC-7738.

In particular embodiments, when the combination is NUC-7738 and pembrolizumab, the NUC-7738 is administered at a dose select from: 1125 mg/m², or 900 mg/m², or 1350 mg/m² on days 1, 8 and 15 of a 21-day cycle and pembrolizumab is administered at 200 mg on day 1 of the 21-day cycle. It may be possible to administer pembrolizumab at 400 mg (Q6w), in which case the NUC-7738 could be administered on days 1, 8, 15, 22, 29 and 35 of a 42-day cycle and 400 mg pembrolizumab given on day 1.

When utilsing an immune checkpoint inhibitor other than pembrolizumab, for example one selected from cemiplimab, dostarlimab, nivolumab, durvalumab, ipilimumab, atezolizumab, and avelumab, the embodiments as for pembrolizumab apply mutatis mutandis.

According to another aspect the present invention provides NUC-7738 for use in the treatment of a proliferative disease, wherein the NUC-7738 is for simultaneous or sequential administeration with an immune oncology agent, such as an immune checkpoint inhibitor as defined herein.

According to another aspect the present invention provides a method of treating a proliferative disease in a subject in need thereof comprising administering to said subject a combination comprising NUC-7738 and an immune oncology agent, such as an immune checkpoint inhibitor as defined herein, wherein the NUC-7738 and immune oncology agent are administered simultaneously or sequentially.

According to another aspect the present invention provides an immune checkpoint inhibitor as defined herein for use in the treatment of a proliferative disease, wherein the immune checkpoint inhibitor is for simultaneous or sequential administeration with NUC-7738 as defined herein.

According to another aspect the present invention provides the use of an immune checkpoint inhibitor in the manufacture of a medicament for treating a proliferative disease in combination with NUC-7738 as defined herein.

In particular embodiments, the cancer is selected from: skin cancer (such as melanoma Merkel cell carcinoma or cutaneous melanoma), lung cancer (including NSCLC), breast cancer, colorectal cancer, renal cancer, liver cancer, thyroid cancer, gastric cancer, pancreatic cancer, head and neck cancer, prostate cancer, kidney cancer, bladder cancer, lymphoma (such as Hodgkin lymphoma), ovarian cancer, cervical cancer and endometrial cancer. In particular embodiments, the cancer is one that is normally treated with an immune checkpoint inhibitor, such as pembrolizumab. Table 1 provides a list of diseases that particular checkpoint inhibitor agents can be used to treat. Suitably, the cancer comprises cancer cells that express high levels of soluble or exosomal PD-L1. Suitably, the cancer comprises cancer cells that express low levels of OX40L.

### “Pharmaceutical Product Comprising NUC-7738”

According to another aspect the present invention provides a pharmaceutical product comprising NUC-7738 and an immune oncology agent.

In a particular embodiment, the immune oncology agent is an immune checkpoint inhibitor, as described herein.

In one embodiment, the pharmaceutical product may comprise a kit of parts comprising separate formulations of NUC-7738 and an immune checkpoint inhibitor.

The separate formulations of NUC-7738 and the immune checkpoint inhibitor may be administered or suitable for administering or adapted from administration sequentially and/or simultaneously.

In another embodiment the pharmaceutical product is a kit of parts which comprises:
a first container comprising NUC-7738, such as NUC-7738 in association with a pharmaceutically acceptable adjuvant, diluent or carrier; and
a second container comprising an immune checkpoint inhibitor such as an immune checkpoint inhibitor in association with a pharmaceutically acceptable adjuvant, diluent or carrier, and
a container means for containing said first and second containers.

In particular embodiments, the pharmaceutical product may comprise a one or more unit dosage forms (e.g. vials, tablets or capsules in a blister pack). In one embodiment, each unit dose comprises only one agent selected from NUC-7738 and the immune checkpoint inhibitor. In another embodiment, the unit dosage form comprises both the NUC-7738 compound and the immune checkpoint inhibitor.

### “Immune Checkpoint Inhibitors”

Immune checkpoint proteins present on immune cells and/or cancer cells [e.g. PD1 (also known as programmed cell death protein 1 and CD279), PD-L1 (also known as programmed death-ligand 1 and CD274), CTLA4 (also known as cytotoxic T-lymphocyte-associated protein 4 and CD152), LAG3 (also known as lymphocyte-activation gene 3 and CD223), TIM-3 (also known as T-cell immunoglobulin mucin-3) and TIGIT (also known as T-cell Immunoreceptor with Ig and

ITIM domains) are molecular targets that have been found to play an important role in regulating anti-tumour immune responses. Inhibitors of these immune checkpoint proteins (e.g. CTLA4, LAG3, PD1, PD-L1, TIM-3, BTLA, OX40, OX40L and/or TIGIT inhibitors) promote an anti-tumour immune response that can be utilised to effectively treat certain forms of cancer.

Reference to immune checkpoint inhibitor herein refers to any immune checkpoint inhibitor including any disclosed herein. Thus, any immune checkpoint inhibitor may be used in the combination therapy defined herein.

In one embodiment, the immune checkpoint inhibitor is selected from a PD-1 inhibitor, a PD-L1 inhibitor, a LAG3 inhibitor, CTLA-4 inhibitor, a TIM-3 inhibitor, a BTLA inhibitor, an OX40 inhibitor, an OX40L inhibitor and/or a TIGIT inhibitor. In a particular embodiment, the immune checkpoint inhibitor is a PD-1 or PD-L1 inhibitor. In particular embodiments, the immune checkpoint inhibitor is an antibody against an immune checkpoint protein, such as an anti-PD-1 antibody, an anti-PD-L1 antibody, an anti-LAG3 antibody, and anti-CTLA-4 antibody, an anti-TIM3 antibody, and anti-BTLA antibody, and anti-OX40 antibody or an anti-OX40L antibody. As used herein the term antibody also included fragments of whole antibodies capable of binding to the target antigen, including Fab, scFV, and the like.

PD-1 is a cell surface receptor protein present on immune cells such as T cells. PD-1 plays an important role in down-regulating the immune system and promoting self-tolerance by suppressing T cell activation. The PD-1 protein is an immune checkpoint that guards against autoimmunity through a dual mechanism of promoting apoptosis (programmed cell death) in antigen specific T cells in lymph nodes, while simultaneously reducing apoptosis in regulatory T cells (anti-inflammatory suppressive T cells).

PD-1 therefore inhibits the immune system. This prevents autoimmune diseases, but it can also prevent the immune system from killing cancer cells.

PD-1 binds two ligands, PD-L1 and PD-L2. PD-L1 is of particular interest as it is highly expressed in several cancers and hence the role of PD-1 in cancer immune evasion is well established. Monoclonal antibodies targeting PD-1 that boost the immune system are approved or are being developed for the treatment of many types of cancer. Many tumour cells express PD-L1, an immunosuppressive PD-1 ligand; it is well documented that inhibition of the interaction between PD-1 and PD-L1 can enhance T-cell responses in vitro and mediate preclinical antitumour activity. This is known as immune checkpoint blockade.

Examples of drugs that target PD-1 include pembrolizumab (Keytruda™), nivolumab (Opdivo™) and cemiplimab (Libtayo™). These drugs have been shown to be effective in treating several types of cancer, including melanoma of the skin, cutaneous squamous cell carcinoma, non-small cell lung cancer, kidney cancer, bladder cancer, head and neck cancers, and Hodgkin lymphoma. They are also being studied for use against many other types of cancer. Examples of drugs in development include BMS-936559 (Bristol Myers Squibb), MGA012 (MacroGenics) and MEDI-0680 (MedImmune).

Examples of drugs that inhibit PD-L1 include atezolizumab (Tecentriq), avelumab (Bavencio) and durvalumab (Imfinzi). These drugs have also been shown to be helpful in treating different types of cancer, including bladder cancer, non-small cell lung cancer, small cell lung cancer, hepatocellular carcinoma, and Merkel cell skin cancer (Merkel cell carcinoma). They are also being studied for use against other types of cancer.

Examples of LAG3 inhibitors include BMS-986016/relatlimab, TSR-033, REGN3767, MGD013 (bispecific DART binding PD-1 and LAG-3), GSK2831781 and LAG525.

Examples of CTLA-4 inhibitors include MDX-010/ipilimumab, AGEN1884, and CP-675,206/tremelimumab.

Examples of TIM-3 inhibitors include sabatolimab (also known as MBG453 (Novartis)), cobolimab (also known as TSR-022 (Tesaro/GlaxoSmithKline)), LY3321367 (Lilly) and BMS-986258 (Bristol-Myers Squibb).

Examples of TIGIT inhibitors include tiragolumab (MTIG7192A; RG6058; Genentech/Roche), AB154 (Arcus Bioscience), MK-7684 (Merck), BMS-986207 (Bristol-Myers Squibb), ASP8374 (Astellas Pharma; Potenza Therapeutics).

Examples of OX40 inhibitors include: MEDI6469 (AstraZeneca) and BMS-986178 (Bristol-Myers Squib).

Examples of OX40-L inhibitors include: SL-279252 (PD1-Fc-OX40L-Shattuck Labs).

Examples of BTLA inhibitors include: INBRX-106 (InhinRX), cudarolimab (also known as IBI 101 (Innovent Biologics)).

In particular embodiments, the immune checkpoint inhibitor is selected from BMS-986016/Relatlimab, TSR-033, REGN3767, MGD013 (bispecific DART binding PD-1 and LAG-3), GSK2831781, LAG525, MDX-010/Ipilimumab, AGEN1884, and CP-675,206/Tremelimumab, pembrolizumab, nivolumab, atezolizumab, avelumab, durvalumab, MBG453, TSR-022, LY3321367, Tiragolumab (MTIG7192A; RG6058), AB154, MK-7684, BMS-986207, ASP8374, MEDI6469. BMS-986178, SL-279252, INBRX-106 and Cudarolimab, or a pharmaceutically acceptable salt or solvate thereof.

In one embodiment, the immune checkpoint inhibitor is an anti-PD-L1 antibody.

In particular embodiments, the immune checkpoint inhibitor is an anti-PD-L1 antibody is selected from the group consisting of: atezolizumab, avelumab and durvalumab.

In one embodiment, the immune checkpoint inhibitor is an anti-PD-1 antibody.

In particular embodiments, the immune checkpoint inhibitor is an anti-PD-1 antibody selected from the group consisting of: nivolumab, pembrolizumab, dostarlimab and cemiplimab.

In a particular embodiment, the immune checkpoint inhibitor is the anti-PD-1 antibody pembrolizumab.

In a particular embodiment, the immune checkpoint inhibitor is the anti-PD-1 antibody nivolumab.

In a particular embodiment, the immune checkpoint inhibitor is the anti-PD-1 antibody cemiplimab.

In a particular embodiment, the immune checkpoint inhibitor is the anti-PD-1 antibody dostarlimab.

In one embodiment, the immune checkpoint inhibitor is an anti-CTLA-4 antibody.

In particular embodiments, the immune checkpoint inhibitor is an anti-CTLA-4 antibody selected from ipilimumab or tremelimumab.

In one embodiment, the immune checkpoint inhibitor is an anti-LAG-3 antibody.

In a particular embodiment, the anti-LAG-3 antibody is relatimab.

In one embodiment, the immune checkpoint inhibitor is an anti-TIGIT antibody.

In a particular embodiment, the anti-TIGIT antibody is tiragolumab.

In one embodiment, the immune checkpoint inhibitor is an anti-OX40 antibody.

In a particular embodiment, the anti-OX40 antibody is selected from MEDI6469 and BMS-986178.

In one embodiment, the immune checkpoint inhibitor is an anti-OX40-L antibody.

In a particular embodiment, the anti-OX40-L antibody is SL-279252.

In one embodiment, the immune checkpoint inhibitor is an anti-BTLA antibody.

In a particular embodiment, the anti-BTLA antibody is selected from INBRX-106 and Cudarolimab.

### Antibodies (Including Antigen-Binding Fragments of Antibodies)

An antibody is an immunoglobulin molecule capable of specific binding to a target, such as a
protein, polypeptide, peptide, carbohydrate, polynucleotide, lipid, or combinations of the foregoing through at least one antigen recognition site, located in the variable domain of the
immunoglobulin molecule. In particular, as used herein, the term “antibody” encompasses intact polyclonal antibodies, intact monoclonal antibodies, multispecific antibodies such as bispecific antibodies generated from at least two intact antibodies, chimeric antibodies, humanised antibodies, human antibodies, any other modified immunoglobulin molecule and any fragments thereof comprising an antigen binding site so long as the antibodies exhibit the desired biological activity. The term embraces whole antibodies (such as IgG1, IgG4 and the like) and antigen binding fragments. The antibody can be from any species. Suitably the antibody is a human antibody.

The antigen-binding site refers to the part of a molecule that binds to all or part of the target antigen. In an antibody molecule it may be referred to as the antibody antigen-binding site and comprises the part of the antibody that specifically binds to all or part of the target antigen. Where an antigen is large, an antibody may only bind to a particular part of the antigen, which part is termed an epitope. An antibody antigen-binding site may be provided by one or more antibody variable domains. Preferably, an antibody antigen-binding site comprises an antibody light chain variable region (VL) and an antibody heavy chain variable region (VH). The invention also encompasses antibody-fragments that comprise an antigen-binding site. Thus, the term “antigen-binding fragment thereof”, when referring to an antibody refers to antibody fragments, such as Fab, Fab′, F(ab′)2, diabodies, Fv fragments and single chain Fv (scFv) mutants that possess an antigen recognition site, and thus, the ability to bind to an antigen. Antigen binding immunoglobulin (antibody) fragments are well known in the art. Such fragment need not have a functional Fc receptor binding site.

### “Treatment of a Proliferative Disease”

The term “proliferative disease” is used herein to refer to an unwanted, uncontrolled and abnormal cellular proliferation, such as, neoplastic or hyperplastic growth, whether in vitro or in vivo. Examples of proliferative conditions include, but are not limited to, benign, pre-malignant and malignant cellular proliferation, including but not limited to, malignant neoplasms and tumours, cancers, leukemias, psoriasis, bone diseases, fibroproliferative diseases (e.g., of connective tissues), and atherosclerosis. Any type of cell may be treated, including but not limited to, lung, colon, breast, ovarian, prostate, liver, pancreas, brain, bladder, kidney, bone, nerves and skin (including melanoma such as cutaneoaus melanoma). The various aspects of the present invention have particular application in the treatment of cancers, particularly human cancers. Suitably, the proliferative disease is one that is normally treated with an immune checkpoint inhibitor, such as pembrolizumab. Table 1 provides a list of diseases that particular checkpoint inhibitor agents can be used to treat. The section entitled “Immune checkpoint inhibitors” also identifies cancer types which the various immune checkpoint inhibitors are proposed for treating. Suitably, the proliferative disease is cancer which comprises cancer cells that express high levels of soluble or exosomal PD-L1. Suitably, the proliferative disease is cancer which comprises cancer cells that express low levels of OX40L.

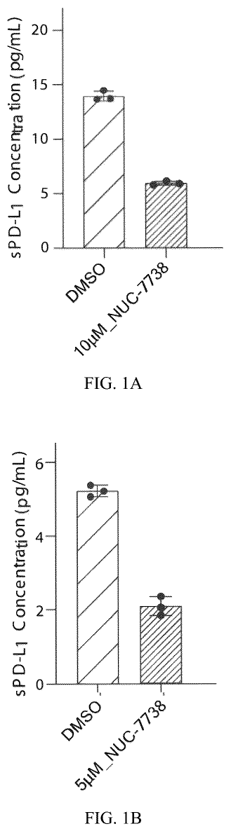

As can be seen from FIG. 1A and FIG. 1B, NUC-7738 reduces soluble PD-L1 in lung and melanoma cell lines which supports the use of NUC-7738 in treating any proliferative disease mediated by or associated with high levels of sPD-L1 or one where sPD-L1 is implicated (e.g. due to its ability to dampen the host immune system). This includes all cancer.

In particular embodiments, the cancer is selected from: skin cancer (such as melanoma Merkel cell carcinoma), lung cancer (including NSCLC), breast cancer, colorectal cancer, renal cancer, liver cancer, thyroid cancer, gastric cancer, pancreatic cancer, head and neck cancer, prostate cancer, kidney cancer, bladder cancer, lymphoma (such as Hodgkin lymphoma), ovarian cancer, cervical cancer and endometrial cancer.

Melanoma is particularly suited for treatment with NUC-7738, alone or in combination with an immune checkpoint inhibitor such as an anti-PD-1 antibody.

Cutaneous melanoma is particularly suited for treatment with NUC-7738, alone or in combination with an immune checkpoint inhibitor, such as an anti-PD-1 antibody, such as pembrolizumab.

“Disease or Condition Mediated by or Associated with High Levels of PD-L1 or Low Levels of OX40L”

Suitably, NUC-7738 can be used to treat a patient with a proliferative disease with high levels of PD-L1 generally, but in particular of soluble PD-L1 and/or exosomal PD-L1.

Suitably, NUC-7738 can be used to treat a patient with a proliferative disease with low OX40-L protein levels. Suitably the sPD-L1, xPD-L1 and/or OX40-L levels are those produced by cancer cells. Suitably the sPD-L1, xPD-L1 and/or OX40-L levels are detected in a blood sample or fraction thereof (e.g. plasma, serum) from the patient. Suitably, the proliferative disease is a cancer and a patient to be treated can be identified by the levels of PD-L1 (e.g. soluble or exosomal PD-L1) or OX40-L in relevant cells or biofluids, and thus the invention provides for the use of NUC-7738 in the treatment of a proliferative disease, such as cancer, associated with expression of high levels of PD-L1 (e.g. soluble or exosomal PD-L1) and/or low levels of OX40-L.

Accordingly, in part, the present invention relates to medical uses of NUC-7738 for the treatment of a proliferative disease mediated by, or associated with, high levels of soluble PD-L1 and/or exosomal PD-L1 and/or low levels of OX40-L. Suitably, the proliferative disease is cancer.

In particular embodiments, the proliferative disease is cancer whose cells express high levels of soluble PD-L1 and/or exosomal PD-L1 and/or low levels of OX40-L. In particular embodiments, the cancer whose cells express high levels of soluble PD-L1 and/or exosomal PD-5 L1 and/or low levels of OX40-L is selected from: skin cancer (such as melanoma Merkel cell carcinoma), lung cancer (including NSCLC), breast cancer, colorectal cancer, renal cancer, liver cancer, thyroid cancer, gastric cancer, pancreatic cancer, head and neck cancer, prostate cancer, kidney cancer, bladder cancer, lymphoma (such as Hodgkin lymphoma), ovarian cancer, cervical cancer and endometrial cancer.

According to one aspect the present invention provides NUC-7738 for use in the treatment of cancer in a subject whose cancer cells express high levels of soluble PD-L1 or exosomal PD-L1 and/or low levels of OX40-L.

According to another aspect the present invention provides the use of NUC-7738 in the manufacture of a medicament for treating cancer in a subject whose cancer cells express high levels of soluble PD-L1 or exosomal PD-L1 and/or low levels of OX40-L, wherein the treatment comprises administering the NUC-7738 in combination with an immune checkpoint inhibitor as defined herein. Optionally, prior to the treating the patient is tested to determine whether they have a cancer whose cancer cells express high levels of soluble PD-L1 and/or high levels of exosomal PD-L1 and/or low levels of OX40-L.

According to another aspect the present invention provides use of NUC-7738 in the manufacture of a medicament for use in a method of treating cancer by reducing the amount of soluble PD-L1 and/or exosomal PD-L1 produced by the cancer cells, wherein the method comprises determining whether a subject has a cancer whose cancer cells express high levels of soluble PD-L1 or exosomal PD-L1, wherein if the subject has a cancer whose cancer cells express high levels of soluble or exosomal PD-L1 the subject is administered a combination of NUC-7738 and an immune checkpoint inhibitor as defined herein.

According to another aspect the present invention provides a method of treating cancer in a subject whose cancer cells express high levels of soluble PD-L1 or exosomal PD-L1, comprising administering to the subject a therapeutically effective amount of NUC-7738 alone or in combination with an immune checkpoint inhibitor.

According to another aspect the present invention provides a method of treating cancer in a subject whose cancer cells express high levels of soluble PD-L1 or exosomal PD-L1, comprising contacting the cancer cells with a therapeutically effective amount of NUC-7738. In particular embodiments of this aspect of the invention the cancer cells are within a subject or patient. Thus, suitably, the cancer cells are contacted with a therapeutically effective amount of NUC-7738 by administering to the subject a therapeutically effective amount of NUC-7738, alone or in combination with an immune checkpoint inhibitor.

According to another aspect the present invention provides use of NUC-7738 in the manufacture of a medicament for use in a method of treating cancer by increasing the amount of OX40-L produced by the cancer cells, wherein the method comprises determining whether a subject has a cancer whose cancer cells express low levels of OX40-L, wherein if the subject has a cancer whose cancer cells express low levels of OX40-L the subject is administered a combination of NUC-7738 and an immune checkpoint inhibitor as defined herein.

According to another aspect the present invention provides a method of treating cancer in a subject whose cancer cells express low levels of OX40-L, comprising administering to the subject a therapeutically effective amount of NUC-7738 alone or in combination with an immune checkpoint inhibitor.

According to another aspect the present invention provides a method of treating cancer in a subject whose cancer cells express low levels of OX40-L, comprising contacting the cancer cells with a therapeutically effective amount of NUC-7738. In particular embodiments of this aspect of the invention the cancer cells are within a subject or patient. Thus, suitably, the cancer cells are contacted with a therapeutically effective amount of NUC-7738 by administering to the subject a therapeutically effective amount of NUC-7738, alone or in combination with an immune checkpoint inhibitor.

In particular embodiments, the level of soluble PD-L1 and/or exosomal PD-L1 in serum of a cancer patient whose cancer cells express high levels of soluble PD-L1 or exosomal PD-L1, is higher than the reference level, by at least 30%, such as by at least 50%, at least 75%, at least 90%, at least 95%, at least 100%, at least 200%, at least 300%, at least 400%, at least 500%, at least 750%, at least 1000%, at least 2000%, of the reference level. The reference level may be one assigned to that for a healthy individual, i.e. one that does not have the disease (e.g. cancer). Such individual may be referred to as having normal or plasma.

The level of sPD-L1 in normal plasma is about 10 pg/mL, whereas in a subject whose cancer cells express high levels of sPD-L1 the level of sPD-L1 in plasma may be about 40 pg/mL to about 200 pg/mL. In particular embodiments, the uses and methods of the invention are for treating a cancer patient that possesses between about 40 pg/mL and 200 μg/mL sPD-L1, such as between about 60 pg/mL and 150 pg/mL or between about 70 pg/mL and 120 pg/mL sPD-L1 in their serum.

In particular embodiments, the level of OX40-L in a sample (e.g. cell or biofluid like serum) from a cancer patient whose cancer cells express low levels of OX40-L, is higher than the reference level, by at least 30%, such as by at least 50%, at least 75%, at least 90%, at least 95%, at least 100%, at least 200%, at least 300%, at least 400%, at least 500%, at least 750%, at least 1000%, at least 2000%, of the reference level. The reference level may be one assigned to that for a healthy individual, i.e. one that does not have the disease (e.g. cancer). Such individual may be referred to as having normal or plasma.

### Reference Level

The reference level can be determined by parallel measurements of soluble PD-L1 and/or exosomal PD-L1 and/or OX40-L in normal cells from the patient, or they can be values determined from historic measurements of wild type/normal cells, e.g. an average wild type level determined with relevant statistical significance from multiple samples. In view of widely recognised interpatient variability, the reference wild type level can be a “normal range”, for example the lowest value in a normal range. Thus, by way of example, if the normal range for total protein in the blood is between 6 and 8.3 grams per decilitre (g/dL). The reference wild type value for protein in the blood could be taken as the 6 g/dL or 8.3g/dL value depending on whether a value lower or higher than the normal was to be detected. In a similar way, the normal range for the amount of soluble PD-L1 and/or exosomal PD-L1 or OX40-L can be determined using routine assessments for any particular cell type and used to identify a “reference value” or “reference level” that can be used to gauge whether the cell expresses normal, low or high levels of soluble PD-L1, and/or exosomal PD-L1, and/or OX40-L and thus whether the patient would benefit from treatment with NUC-7738 according to the methods of the invention. The person skilled in the art is able to identify the appropriate reference value to use to determine whether the cancer expresses high levels of soluble PD-L1 and/or high levels of exosomal PD-L1 and/or low levels of OX40-L. In particular embodiments, the wild-type value or level can be used as the reference value or level. In other embodiments, the reference value or level is a value or level established from statistical assessment of multiple samples (e.g. from healthy individuals and/or those with a disease, such as cancer) that can be used to assign a test subject to a category (e.g. healthy or one with high levels of sPDL-1) with a certain degree of statistical confidence (e.g. 95% confidence level). As described above, the person of skill in the art is able to determine a reference/cut-off value or level using established and standard techniques.

In particular embodiments, the NUC-7738 reduces the amount of soluble PD-L1 and/or exosomal PD-L1 produced by an affected cell (e.g. cancer cell) by at least 25%, such as at least 30%, at least 40%, at least 50%, at least 70%, at least 80% or at least 90% relative to the level prior to treatment.

In a particular embodiment, treatment with NUC-7738 reduces soluble PD-L1 or exosomal PD-L1 levels by at least about 25% compared the levels obtained in the absence of NUC-7738 treatment.

In particular embodiments, treatment with NUC-7738 reduces soluble PD-L1 or exosomal PD-L1 levels by approximately 10 to 80% compared the levels obtained in the absence of NUC-7738 treatment.

In particular embodiments, the NUC-7738 reduces the amount of soluble PD-L1 and/or exosomal PD-L1 present in the serum to levels that are substantially the same as normal/reference levels. By substantially the same we mean±10%.

In particular embodiments, treatment with NUC-7738 reduces soluble PD-L1 or exosomal PD-L1 levels by 2- to 10-fold compared the levels obtained in the absence of NUC-7738 treatment.

In particular embodiments, the NUC-7738 increases the amount of OX40-L produced by an affected cell (e.g. cancer cell) by at least 25%, such as at least 30%, at least 40%, at least 50%, at least 70%, at least 80% or at least 90% relative to the level prior to treatment.

In a particular embodiment, treatment with NUC-7738 increases the OX40-L levels by at least about 25% compared the levels obtained in the absence of NUC-7738 treatment.

In particular embodiments, treatment with NUC-7738 increases the OX40-L levels by approximately 10 to 80% compared the levels obtained in the absence of NUC-7738 treatment.

In particular embodiments, the NUC-7738 increases the OX40-L level present in the serum to a level that is substantially the same as normal/reference levels. By substantially the same we mean±10%.

In particular embodiments, treatment with NUC-7738 increases OX40-L levels by 2- to 10-fold compared the levels obtained in the absence of NUC-7738 treatment.

The various aspects of the invention are based upon the finding that NUC-7738 is able to reduce the amount of soluble PD-L1 and/or exosomal PD-L1 produced by cancer cells and/or increase the amount of OX40-L produced by cancer cells. PD-L1 and OX40-L are known to mask the cancer cell from the immune system and soluble PD-L1 in particular is a biomarker of poor prognosis and resistance to immune checkpoint inhibitors. Accordingly, the ability of NUC-7738 to reduce the amount of soluble PD-L1 and/or exosomal PD-L1 produced by the cancer cells and/or increase the level of OX40-L produced by cancer cells provides new clinical opportunities for treating and managing cancers in patients. For example, by removing or lessening the camouflage that the cancer cell has from the immune system, thus allowing the host immune system to recognise and attack, include kill, the cancer cell. The ability of NUC-7738 to reduce the levels of soluble PD-L1 and/or exosomal PD-L1 and/or increase the level of OX40-L produced by cancer cells will serve to enhance the effectiveness of an immune oncology agent, including immune checkpoint inhibitors, which provides for the ability to treat a cancer patient with a combination of NUC-7738 and an immune oncology agent. When the immune oncology agent is an immune checkpoint inhibitor, as described herein, it may also facilitate the use of a lower dose of the checkpoint inhibitor thus lessening the toxic effect of such agent. Certain cancer patients also develop resistance to immune checkpoint inhibitors which is often associated with an increase in soluble PD-L1 levels. The ability of NUC-7738 to target and reduce the amount of soluble PD-L1 or exosomal PD-L1 is predicted to remove the resistance/block and thus allow the patient to be re-treated with the same or an alternate immune checkpoint inhibitor. NUC-7738 can therefore also be used to treat patients that are or have developed resistance to an immune checkpoint inhibitor.

Thus, in particular embodiments of any of the aspects of the invention the patient or subject is one that is or has developed resistance to an immune checkpoint inhibitor. Suitably, such person is one where established doses of the immune checkpoint inhibitor do not provide recognised clinical signals of efficacy.

Suitably the subject/patient will have previously shown clinical signals of efficacy, such as maintenance or reduction in cancer mass or reduction in markers of cancer, progression free survival, but the effectiveness of the immune checkpoint inhibitor has then reduced or ceased. This is an indication that the cancer cells have become resistant to the agent.

Suitably a subject/patient that is resistant to (has pre-existing resistance to) an immune checkpoint inhibitor will have markers indicating pre-existing resistance to immune checkpoint inhibitor therapy. Various markers of resistance to immune-checkpoint therapy have been identified, including: low tumour mutational burden/MSS tumour, low tumour infiltrates, high exo-PD-L2/sPD-L1 and low OX40L.

### Selection of Patients

The inventors' finding that NUC-7738 is able to alter the expression of mRNA encoding soluble/exosomal PD-L1, and reduce the amount of soluble PD-L1 and exosomal PD-L1, and/or increase the amount of OX40L, makes possible a number of methods by which it is possible to determine whether a particular patient is likely to benefit from receiving NUC-7738, either alone or in combination with an immune checkpoint inhibitor as described herein.

Thus, according to another aspect the present invention provides a method of determining whether a patient will benefit from treatment with NUC-7738, the method comprising: determining the level of soluble PD-L1 and/or exosomal PD-L1 and/or OX40-L in a biological sample from the patient; wherein if the level of soluble PD-L1 and/or exosomal PD-L1 produced by the cancer cells in the biological sample is elevated and/or the level of OX40-L L1 produced by the cancer cells in the biological sample is reduced compared to wild-type or a reference value the patient will benefit from treatment with NUC-7738.

Suitably, the biological sample is a blood sample or a fraction therefrom (e.g. plasma or serum).

In particular embodiments of this aspect, the amount or level of soluble-PD-L1 or exosomal PD-L1 is determined and if such amount or level is elevated compared to wild-type or a reference value the patient will benefit from treatment with NUC-7738.

In one particular embodiment, the level of soluble PD-L1 is determined.

In one particular embodiment, the level of exosomal PD-L1 is determined.

In another particular embodiment, the levels of soluble PD-L1 and exosomal PD-L1 are determined.

In a particular embodiments of this aspect, the amount or level of OX40-L is determined and if such amount or level is reduced compared to wild-type or a reference value the patient will benefit from treatment with NUC-7738.

In a particular embodiment, the levels of soluble PD-L1 and OX-40-L are determined.

In another particular embodiment, the levels of exosomal PD-L1 and OX-40-L are determined.

In another particular embodiment, the levels of soluble PD-L1 and exosomal PD-L1 and OX-40-L are determined.

### Method of Determining a Suitable Treatment Regimen

According to another aspect the present invention provides a method of determining a suitable treatment regimen for a patient with a proliferative disease, such as cancer, the method comprising: assaying a biological sample from the patient for the level of soluble PD-L1 and/or exosomal PD-L1 and/or OX40-L; wherein the presence of an elevated level of the determined soluble PD-L1 and/or an elevated level of the determined exosomal PD-L1 and/or a reduced level of the determined OX40-Lrelative to wild type or a reference value in the biological sample indicates that a suitable treatment regimen will comprise treatment of the patient with NUC-7738.

The person of skill in the art will appreciate that the method of determining a suitable treatment regimen for a patient includes a method of selecting a patient for treatment.

According to another aspect the present invention provides a method of determining whether a patient with cancer will benefit from treatment with NUC-7738, the method comprising: determining the level of soluble PD-L1 and/or exosomal PD-L1 and/or OX40-L in a biological sample from the patient; wherein if the level of soluble PD-L1 and/or exosomal PD-L1 in the biological sample is elevated and/or the level of OX40-L is reduced compared to wild-type or a reference value the patient will benefit from treatment with NUC-7738. Suitably, the soluble PD-L1 and/or exosomal PD-L1 and/or OX40-L in the biological sample has been produced by the cancer cells.

According to another aspect the present invention provides a method of treating a patient with a proliferative disease, such as cancer, the method comprising: assaying a biological sample from the patient for the level of soluble PD-L1 and/or exosomal PD-L1 and/or OX40-L; wherein if level of the determined soluble PD-L1 and/or exosomal PD-L1 is higher than wild type or a reference value and/or if level of the determined OX40-L is lower than wild type or a reference value the patient is administered an effective amount of NUC-7738.

Suitably the level of the PD-L1 (soluble and/or exosomal) and/or OX40-L can be determined quantitatively using a suitable immunoassay.

Suitably, the biological sample is a blood sample or a fraction therefrom (e.g. plasma or serum).

The skilled person will appreciate that there are many suitable examples of biological samples that may be used in embodiments of the invention such as those set out above. Suitably such a sample may include cancer cells from diseased tissue or organ (e.g. skin, kidney, brain, liver) or a liquid sample (such as blood or a fraction thereof). A suitable biological sample may be a tissue sample, such as a sample for use in histology. Cells in such samples may be directly assessed for their level of soluble PD-L1 and/or exosomal PD-L1 and/or OX40-L, such as those set out above. A particularly suitable sample is a blood sample or blood fraction thereof, such as plasma or serum.

Techniques for the investigation of the levels of biomarkers are frequently used in the context of clinical assessments (such as for diagnostic or prognostic purposes) and their use will be familiar to those required to practice them in the context of the present invention. Merely by way of example, in samples containing proteins the presence of soluble PD-L1 and/or exosomal PD-L1 and/or OX-40-Lmay be assessed by suitable techniques using antibodies that react with the marker (e.g. PD-L1 or OX40-L as appropriate) in question. Examples of suitable samples include histology samples (where the presence of the markers may be visualised by suitable immunocytochemistry techniques).

### Particular Embodiments:

1. NUC-7738 for use in the treatment of a proliferative disease by reducing the amount of extra-cellular PD-L1 protein and/or increasing the amount of OX40-L protein produced by the proliferative disease cells.

2. NUC-7738 for use according to embodiment 1, wherein the extra-cellular PD-L1 protein is soluble PD-L1 and/or exosomal PD-L1.

3. NUC-7738 for use according to embodiment 2, wherein the treatment with NUC-7738 causes a reduction in the level of soluble PD-L1 and/or exosomal PD-L1 and/or an increase in level of OX40-L protein produced by the proliferative disease cells.

4. NUC-7738 for use according to any one of embodiments 1 to 3, wherein administration of NUC-7738 to a patient enhances the patient's immune response against the proliferative disease.

5. NUC-7738 for use according to any one of embodiments 1 to 4, wherein the treatment of the proliferative disease arises through adaptive immunity (e.g. cellular immunity and/or humoral immunity).

6. NUC-7738 for use according to any one of the preceding embodiments, wherein the proliferative disease is cancer.

7. NUC-7738 for use as an immune-sensitiser in the treatment of cancer.

8. NUC-7738 for use according to embodiment 6 or 7, wherein the cancer is selected from:
melanoma (including cutaneous melanoma), lung cancer (including NSCLC), breast cancer, colorectal cancer, renal cancer, liver cancer, thyroid cancer, gastric cancer, pancreatic cancer, head and neck cancer, prostate cancer, bladder cancer, lymphoma, ovarian cancer, cervical cancer and endometrial cancer.

9. NUC-7738 for use according to any one of the preceding embodiments, wherein the treatment comprises administration of NUC-7738 in combination with an immune oncology agent such as an immune checkpoint inhibitor, an antibody therapy, an adoptive cell therapy such as CAR-T therapy, or a cancer vaccine.

10. NUC-7738 for use according to embodiment 6, wherein the treatment comprises administration of NUC-7738 in combination with an immune checkpoint inhibitor.

11. NUC-7738 for use according to embodiment 6, wherein the immune checkpoint inhibitor is selected from a PD-1 inhibitor, a PD-L1 inhibitor, a LAG-3 inhibitor, CTLA-4 inhibitor, a TIM-3 inhibitor, a TIGIT inhibitor, an OX40 inhibitor, an OX40-L inhibitor or a BTLA inhibitor.

12. NUC-7738 for use according to any one of the preceding embodiments, wherein the immune checkpoint inhibitor is an anti-PD-L1 antibody, an anti-PD-1 antibody, an anti-CTLA-4 antibody, an anti-LAG-3 antibody, an anti-TIGIT antibody, an anti-OX40 antibody, an anti-OX40-L antibody or an anti-BTLA antibody.

13. NUC-7738 for use according to embodiment 12, wherein the anti-PD-L1 antibody is selected from the group consisting of: atezolizumab, avelumab and durvalumab.

14. NUC-7738 for use according to embodiment 12, wherein the anti-PD-1 antibody is selected from the group consisting of: nivolumab, pembrolizumab, dostarlimab and cemiplimab.

15. NUC-7738 for use according to embodiment 12, wherein the anti-PD-1 antibody is pembrolizumab.

16. NUC-7738 for use according to embodiment 12, wherein the anti-CTLA-4 antibody is ipilimumab or tremelimumab.

17. NUC-7738 for use according to embodiment 12, wherein the anti-LAG-3 antibody is relatimab.

18. NUC-7738 for use according to embodiment 12, wherein the anti-TIGIT antibody is tiragolumab.

19. NUC-7738 for use according to embodiment 12, wherein the anti-BTLA antibody is cudarolimab.

20. NUC-7738 for use according to any one of the preceding embodiments, wherein the proliferative disease is cancer, and the cancer cells express high levels of soluble PD-L1 protein and/or high levels of exosomal PD-L1 protein and/or low levels of OX40L protein.

21. NUC-7738 for use according to embodiment 20, wherein the treatment with NUC-7738 reduces the amount of extra-cellular PD-L1 protein produced by the cancer cells.

22. NUC-7738 for use according to embodiment 21, wherein the extra-cellular PD-L1 is selected from soluble PD-L1 protein or exosomal PD-L1 protein.

23. NUC-7738 for use according to embodiment 20, wherein the treatment with NUC-7738 increases the amount of OX40-L protein produced by the cancer cells.

24. NUC-7738 for use according to any one of the preceding embodiments, wherein the patient has previously received treatment with an immune checkpoint inhibitor, optionally wherein said treatment has been stopped.

25. NUC-7738 for use according to embodiment 24, wherein the previous treatment with an immune checkpoint inhibitor was stopped due to toxicity, relapse or the cancer becoming resistant to the previous treatment.

26. NUC-7738 for use according to any one of embodiments, wherein the treatment with NUC-7738 reduces soluble or exosomal PD-L1 levels by at least about 25% compared the levels obtained in the absence of NUC-7738 treatment.

27. NUC-7738 for use according to any one of the preceding embodiments, wherein the NUC-7738 is administered at a weekly dose between about 300 mg/m² and 1600 mg/m², such as at a weekly dose of between 500 mg/m² and 1150 mg/m², or a weekly dose between 900 mg/m² and 1350 mg/m².

28. NUC-7738 for use according to any one of embodiments 10 to 27, wherein the immune checkpoint inhibitor for use in combination with the NUC-7738 is administered in a dose that is 50% or less than the standard monotherapy dose for the agent. 298. A method of treating a proliferative disease by reducing the amount of extra-cellular PD-L1 protein and/or increasing the amount of OX40-L protein produced by the diseased cells in a patient comprising administering a therapeutically effective amount of NUC-7738 to a patient in need thereof.

30. The method according to embodiment 29, wherein the proliferative disease is cancer.

31. A method of potentiating the immune response to a cancer, the method comprising administering to a patient in need of such treatment a therapeutically effective amount of NUC-7738, alone or in combination with an immune oncology agent.

33. The method according to embodiment 30 or 31, wherein the cancer is selected from: melanoma (including cutaneous melanoma), lung cancer (including NSCLC), breast cancer, colorectal cancer, renal cancer, liver cancer, thyroid cancer, gastric cancer, pancreatic cancer, head and neck cancer, prostate cancer, bladder cancer, lymphoma, ovarian cancer, cervical cancer and endometrial cancer.

34. The method according to any one of embodiments 29 to 32, wherein the extra-cellular PD-L1 is soluble PD-L1 protein and/or exosomal PD-L1 protein.

34. The method according to any one of embodiments 30 to 33, wherein the administration of NUC-7738 decreases the amount of extra-cellular PD-L1 protein and/or increases the amount of OX40-L protein produced by the cancer cells.

35. The method according to any one of embodiments 31 to 34, wherein the immune oncology agent is an immune checkpoint inhibitor, an antibody therapy, an adoptive cell therapy or a cancer vaccine.

36. The method according to embodiment 35, wherein immune oncology agent is an immune checkpoint inhibitor.

37. The method according to embodiment 36, wherein the immune checkpoint inhibitor is selected from a PD-1 inhibitor, a PD-L1 inhibitor, a LAG-3 inhibitor, CTLA-4 inhibitor, a TIM-3 inhibitor, a TIGIT inhibitor, an OX40 inhibitor, an OX40-L inhibitor or a BTLA inhibitor.

38. The method according to embodiment 37, wherein the immune checkpoint inhibitor is an anti-PD-L1 antibody, an anti-PD-1 antibody, an anti-CTLA-4 antibody, an anti-LAG-3 antibody, an anti-TIGIT antibody, an anti-OX40 antibody, an anti-OX40-L antibody or an anti-BTLA antibody.

39. The method according to embodiment 38, wherein the anti-PD-L1 antibody is selected from the group consisting of: atezolizumab, avelumab and durvalumab.

40. The method according to embodiment 38, wherein the anti-PD-1 antibody is selected from the group consisting of: nivolumab, pembrolizumab, dostarlimab and cemiplimab.

41. The method according to embodiment 38, wherein the anti-PD-1 antibody is pembrolizumab.

42. The method according to embodiment 38, wherein the anti-CTLA-4 antibody is ipilimumab or tremelimumab.

43. The method according to embodiment 38, wherein the anti-LAG-3 antibody is relatimab.

44. The method according to embodiment 38, the anti-TIGIT antibody is tiragolumab.

45. The method according to embodiment 38, wherein the anti-BTLA antibody is cudarolimab.

46. The method according to any one of embodiments 29 to 45, wherein the proliferative disease is cancer and the cancer cells express high levels of soluble PD-L1 protein and/or high levels of exosomal PD-L1 protein and/or low levels of OX40L protein.

47. The method according to any one of embodiments 29 to 46, wherein the patient has previously received treatment with an immune checkpoint inhibitor, optionally wherein said treatment has been stopped.

468. The method according to embodiment 47, wherein the previous treatment with an immune checkpoint inhibitor was stopped due to toxicity, relapse or the cancer becoming resistant to the previous treatment.

49. The method according to any one of embodiments 29 to 48, wherein the treatment with NUC-7738 reduces soluble or exosomal PD-L1 levels by at least about 25% compared the levels obtained in the absence of NUC-7738 treatment.

50. The method according to any one of embodiments 29 to 48, wherein the NUC-7738 is administered at a weekly dose between about 300 mg/m² and 1600 mg/m², such as at a weekly dose of between 500 mg/m² and 1150 mg/m², or a weekly dose between 900 mg/m² and 1350 mg/m².

51. The method according to any one of embodiments 29 to 48, wherein the immune checkpoint inhibitor for use in combination with the NUC-7738 is administered in a dose that is 50% or less than the standard monotherapy dose for the agent.

520. A combination comprising NUC-7738 and an immune oncology agent.

53. The combination according to embodiment 52, wherein the immune oncology agent is an immune checkpoint inhibitor, an antibody therapy, a cancer vaccine or an adoptive cell therapy such as CAR-T cell.

54. The combination according to embodiment 53, wherein the immune checkpoint inhibitor is an anti-PD-L1 antibody, an anti-PD-1 antibody, an anti-CTLA-4 antibody, an anti-LAG-3 antibody, an anti-TIGIT antibody, an anti-OX40 antibody, an anti-OX40-L antibody or an anti-BTLA antibody.

55. The combination according to embodiment 53 or 54, wherein the immune checkpoint inhibitor is selected from the group consisting of: BMS-986016/relatlimab, TSR-033, REGN3767, MGD013 (bispecific DART binding PD-1 and LAG-3), GSK2831781, LAG525, MDX-010/ipilimumab, AGEN1884, and CP-675,206/tremelimumab, pembrolizumab, nivolumab, atezolizumab, avelumab, durvalumab, MBG453, TSR-022, LY3321367, tiragolumab (MTIG7192A; RG6058), AB154, MK-7684, BMS-986207, ASP8374, MEDI6469. BMS-986178, SL-279252, INBRX-106 and cudarolimab, or a pharmaceutically acceptable salt or solvate thereof.

56. The combination according to any one of embodiments 52 to 55, for use in the treatment of a proliferative disease, such as cancer.

57. A kit of parts which comprises:
a first container comprising NUC-7738, such as NUC-7738 in association with a pharmaceutically acceptable adjuvant, diluent or carrier; and
a second container comprising an immune oncology agent such as an immune checkpoint inhibitor in association with a pharmaceutically acceptable adjuvant, diluent or carrier, and
a container means for containing said first and second containers.

58. The kit of parts according to embodiment 57, wherein the immune checkpoint inhibitor is selected from the group consisting of: BMS-986016/relatlimab, TSR-033, REGN3767, MGD013 (bispecific DART binding PD-1 and LAG-3), GSK2831781, LAG525, MDX-010/ipilimumab, AGEN1884, and CP-675,206/tremelimumab, pembrolizumab, nivolumab, atezolizumab, avelumab, durvalumab, MBG453, TSR-022, LY3321367, tiragolumab (MTIG7192A; RG6058), AB154, MK-7684, BMS-986207, ASP8374, MEDI6469. BMS-986178, SL-279252, INBRX-106 and cudarolimab, or a pharmaceutically acceptable salt or solvate thereof.

59. An immune checkpoint inhibitor for use in the treatment of a proliferative disease such as cancer, wherein the immune checkpoint inhibitor is for administeration with NUC-7738, optionally wherein the the immune checkpoint inhibitor and NUC-3378 are administered simultaneously or sequentially.

60. The immune checkpoint inhibitor for use according to embodiment 59, wherein the immune checkpoint inhibitor is selected from the group consisting of: BMS-986016/relatlimab, TSR-033, REGN3767, MGD013 (bispecific DART binding PD-1 and LAG-3), GSK2831781,

LAG525, MDX-010/ipilimumab, AGEN1884, and CP-675,206/tremelimumab, pembrolizumab, nivolumab, atezolizumab, avelumab, durvalumab, MBG453, TSR-022, LY3321367, tiragolumab (MTIG7192A; RG6058), AB154, MK-7684, BMS-986207, ASP8374, MEDI6469. BMS-986178, SL-279252, INBRX-106 and cudarolimab, or a pharmaceutically acceptable salt or solvate thereof.

61. A method of determining whether a patient will benefit from treatment with NUC-7738, the method comprising: determining the level of soluble PD-L1 and/or exosomal PD-L1 and/or OX40-L in a biological sample from the patient; wherein if the level of soluble PD-L1 and/or exosomal PD-L1 in the biological sample is elevated and/or the level of OX40-L protein in the biological sample is reduced compared to a reference value the patient will benefit from treatment with NUC-7738.

62. The method according to embodiment 61, wherein the biological sample is a blood sample or a fraction therefrom (e.g. plasma or serum).

63. The method according to embodiment 61 or 62, wherein the patient has a cancer selected from the group consisting of: melanoma, lung cancer (including NSCLC), breast cancer, colorectal cancer, renal cancer, liver cancer, thyroid cancer, gastric cancer, pancreatic cancer, head and neck cancer, prostate cancer, bladder cancer, lymphoma, ovarian cancer, cervical cancer and endometrial cancer.

64. The method according to any one of embodiments 61 to 63, wherein the patient is one that is resistant to or has developed resistance to an immune checkpoint inhibitor.

65. NUC-7738 for use in the treatment of cancer in combination with an immune oncology agent, such as an immune checkpoint inhibitor.

66. NUC-7738 for use according to embodiment 65, wherein the cancer is a solid tumour.

67. NUC-7738 for use according to embodiment 65 or 66, wherein the cancer is melanoma, such as cutaneous melanoma.

68. NUC-7738 for use according to any one of embodiments 65 to 67, wherein the cancer is one that is normally treated with an immune checkpoint inhibitor, such as pembrolizumab.

69. NUC-7738 for use according to any one of embodiments 65 to 68, wherein the cancer comprises cancer cells which express high levels of soluble PD-L1 protein and/or high levels of exosomal PD-L1 protein and/or low levels of OX40L protein.

70. NUC-7738 for use according to any one of embodiments 65 to 69, wherein the treatment is of a patient with the cancer and the patient has received one or more prior treatments for the cancer, optionally wherein the treatment has stopped.

71. NUC-7738 for use according to embodiment 70, wherein the treatment was stopped due to toxicity, relapse or the cancer becoming resistant to the previous treatment.

72. NUC-7738 for use according to any one of embodiments 65 to 71, wherein the immune oncology agent is pembrolizumab, wherein the NUC-7738 is administered on days 1, 8 and 15 of a 21 day cycle, optionally at 1125 mg/m², and the pembrolizumab is administered at a 200 mg dose on day 1 of the 21-day cycle.

73. NUC-7738 for use according to any one of embodiments 65 to 72, wherein the immune oncology agent is selected from pembrolizumab, cemiplimab, dostarlimab, nivolumab, durvalumab, ipilimumab, atezolizumab, and avelumab.

74. NUC-7738 for use according to any one of embodiments 65 to 73, wherein the immune oncology agent is pembrolizumab.

75. NUC-7738 for use according to any one of embodiments 65 to 74, wherein the NUC-7738 and immune oncology agent are administered in a 21 day or 42 day treatment cycle.

76. NUC-7738 for use according to any one of embodiments 65 to 75, wherein the NUC-7738 is administered at a weekly dose between about 300 mg/m² and 1600 mg/m², such as at a weekly dose of between 500 mg/m² and 1150 mg/m², or a weekly dose between 900 mg/m² and 1350 mg/m².

77. NUC-7738 for use according to embodiment 76, wherein the NUC-7738 is administered at a weekly dose selected from: 900 mg/m², 1125 mg/m² and 1350 mg/m².

78. NUC-7738 for use according to any one of embodiments 65 to 77, wherein the pembrolizumab is administered at a 200 mg dose once every three weeks (Q3W).

79. NUC-7738 for use according to any one of embodiments 65 to 78, wherein the immune oncology agent is pembrolizumab, wherein the NUC-7738 is administered on days 1, 8 and 15 of a 21 day cycle, optionally at 1125 mg/m², and the pembrolizumab is administered at a 200 mg dose on day 1 of the 21-day cycle.

80. NUC-7738 for use according to any one of embodiments 65 to 77, wherein the pembrolizumab is administered at a 400 mg dose once every six weeks (Q6W).

81. NUC-7738 for use according to embodiment 80, wherein the immune oncology agent is pembrolizumab, wherein the NUC-7738 is administered on days 1, 8, 15, 22, 29 and 35 of a 42 day cycle, optionally at 1125 mg/m², and the pembrolizumab is administered at a 400 mg dose on day 1 of the 42-day cycle.

82. NUC-7738 for use according to any one of embodiments 65 to 81, wherein the treatment is of a patient with the cancer and the patient has previously received treatment with an immune checkpoint inhibitor, optionally wherein said treatment has been stopped.

83. NUC-7738 for use according to embodiment 82, wherein the previous treatment was administration of an immune checkpoint inhibitor which treatment was stopped due to toxicity, relapse or the cancer becoming resistant to the previous treatment.

The invention will now be further described with reference to the following Examples.

### EXAMPLES

Two sets of experiments are described in Examples 1-4. In the first instance, samples of plasma were collected from patients before and after treatment with NUC-7738 and then prepared for analysis as described below. In the second instance, A375 melanoma or A549 lung cancer cell lines were cultured and then exposed to NUC-7738 in vitro at doses and times described below. Thereafter, protein and RNA was extracted and analysed.

### Methods:

Patients were treated with NUC-7738 on days 1 and 8 of a 14-day cycle. Patient plasma samples were collected pre-dose, 24 hr after NUC-7738 dosage and the 24 hours post Cycle 2 (that is the second occasion at which patients received treatment).

For Extracellular Vesicle (EV, exosome) preparation, cells were cultured in T175 Nunc™ EasYFlask™ Cell Culture Flasks (ThermoFisher Cat #159910), approximately 50×10⁶ cells. Cells were grown in DMEM, high glucose, GlutaMAX™ Supplement (ThermoFisher Cat #10566016) supplemented with 5% exosome free FBS and 1% Penicillin-Streptomycin (ThermoFisher Cat #15070063).

For sPDL1 analysis, a similar process was undertaken. Cells were grown in Corning® 100 mm TC-treated Culture Dish with DMEM, high glucose, GlutaMAX™ Supplement (ThermoFisher Cat #10566016) supplemented with 10% FBS and 1% Penicillin-Streptomycin (ThermoFisher Cat #15070063), with approximately 2×10⁶ cells.

A375 melanoma cell lines and A549 lung cancer cell lines were treated with solvent-control DMSO or NUC-7738 (10 uM) for 72 hours, after which media supernatants were collected for either soluble PD-L1 analysis or EV isolation. In addition, adherent cells were collected for RNA and protein isolation.

### Sample Preparation for PD-LI Soluble Assay.

Patient plasma samples were centrifuged at 3000×g for 15 minutes to remove cellular debris. Supernatant was aliquoted in 250 μL measures and stored in −80° C. freezers. In-vitro soluble PD-L1 samples were prepared by collecting media supernatant, at the end of the stated time point they were centrifuged at 3000×g for 15 minutes to remove cellular. Furthermore, the media was concentrated using Vivaspin protein concentrator spin columns at the 3 kDa molecular weight cut-off (Cytiva Cat. #28932358). Upon optimisation, 60 minutes of centrifugation at 4000× g achieved 8× concentration factor. The concentrated samples were aliquoted and stored at −80° C.

Exosome Isolation from In-Vitro and Plasma Samples

Patient plasma samples were centrifuged at 3000×g for 15 minutes to remove cellular debris. Then, the supernatant was aliquoted in 250 μL measures and stored in−80° C. freezers. For exosomal isolation ExoQuick® ULTRA EV Isolation Kit for Serum and Plasma (System Biosciences Cat #EQULTRA-20A-1) according to manufacturer's protocol. In brief, 250 μL of plasma samples free of cellular debris was used and 67 μL ExoQuick Solution was added, followed by a 30 minute incubation period at 4° C. After that, the plasma sample and ExoQuick Solution mixture were centrifuged at 3000×g for 10 minutes. Then, carefully without disturbing the pellet, all of the supernatant was aspirated off, making sure no residual traces remained. Next, the pellet was resuspended in Buffer B provided, followed by the addition of Buffer A. The samples was homogenised in the buffer by carefully pipetting up and down until no clumps of the pellet were seen. The resin purification columns were conditioned by removing the storage buffer and washing it with Buffer B. The samples were added to the conditioned purification column and centrifuged at 1000×g for 30 seconds. The eluted samples were quantified for protein concentration using Qubit™ Protein and Protein Broad Range (BR) Assay Kits (Thermofisher Cat #A50668). Samples were then aliquoted according to the required amounts for the downstream applications and stored −80° C.

Isolation of exosomes from in-vitro samples involved growing cells in 5 separate T175 Nunc™ EasYFlask™ Cell Culture Flasks (ThermoFisher Cat #159910) which approximated to 50×10⁶ cells. Cells were grown in DMEM, high glucose, GlutaMAX™ Supplement (ThermoFisher Cat #10566016) in addition, this was supplemented with exosome free FBS. Each T175 flask contained 40 mL of media and was pooled per treatment, totalling 200 mL, the supernatant media was spun for 15 minutes at 4000× g to remove any cellular debris. Furthermore the media was concentrated to 5 mL using Centricon Plus-70 Centrifugal Filter at 100 kDa cut-off (Merck, Cat #UFC710008). The exosomes were than isolated using the ExoQuick® ULTRA EV Isolation Kit for Tissue Culture Media (System Biosciences Cat #EQULTRA-20TC-1) as per manufacturer protocol. The ExoQuick Ultra exosome isolation protocol for tissue culture remains similar to plasma. One will add 1 mL ExoQuick Solution to 5 mL of media concentrated sample followed by overnight incubation.

### Soluble PD-LI Quantification Using Sandwich ELISA

For quantitative measurement of soluble PD-L1 in patient and in-vitro samples Quantikine® ELISA Human/Cynomolgus Monkey PD-L1/B7-H1 Immunoassay (R&D Systems Cat. #DB7H10) was used as per manufacturers recommendation. Samples were prepared as detailed above; 100 μL of patient plasma sample and 100 μL of concentrated media sample from in-vitro were used per well. Plasma samples were done in two technical replicates and in-vitro samples in three biological and two technical replicates.

In brief, the protocol included the preparation of the standard curve from the Human/Cynomolgus Monkey PD-L1/B7-H1 protein standard ranging from 0 to 1600 μg/mL. Serial dilution was carried out using the provided Calibrator Diluent RD5-33 (diluted 1:3). 50 μL of Assay Diluent RD1-41 was added to each well, followed by 100 μL of standard, control, or sample. The samples were incubated for 2 hours at room temperature orbital microplate shaker set at 500 rpm. At the end of the incubation, each well was washed four times using the wash buffer provided. The samples were further incubated with 200 μL of Human/Cynomolgus Monkey B7-H1 conjugate for 2 hours at room temperature orbital microplate shaker set at 500 rpm. Washing steps were repeated, followed by adding 200 μL of Substrate Solution to each well. After 30 minutes of incubation, protected from light, 50 μL of Stop Solution was added to each well. The wells were read using a microplate reader set to 450 nm and 540 nm for wavelength correction to determine the optical density.

Analysis of the results included subtracting the 540 nm readings from 450 nm reading and subtracting blank, diluent only, sample readings. Standard curve generated using four-parameter logistic (4-PL) curve-fit. The PD-L1 concentration in samples was then interpolated using the standard curve. For the in-vitro samples, 8× concentration factor was taken into consideration; therefore, the final concentration was divided by 8.

### Measuring of PD-L1 Protein Expression in Isolated Exosomes Using Capillary-Based Immune Probing.

Analysis of PD-L1 expression in exosomes isolated from plasma samples was carried out using automated western blotting JESS system (BioTechne, ProteinSimples). The 12-230 kDa Separation Module of 25 capillaries kit (ProteinSimple, Cat #SM-W004) was used per manufacture protocol. Briefly, samples were prepared at the appropriate concentration using the kit master-mix and sample dilution buffer containing DT, samples were then denatured for 5 minutes at 95° C. All samples, primary antibodies and secondary antibodies were loaded into the plate provided. Anti-CD81 antibody [M38] (Abcam, Cat #ab79559) diluted at 1:100 and PD-L1 (E1L3N®) XP® Rabbit mAb (Cell Signalling, Cat #13684) diluted at 1:50 were used. Secondary antibodies used were from the anti-mouse chemiluminescence (ProteinSimple, Cat #DM002) and anti-rabbit near-infrared (NIR) (ProteinSimple, Cat #DM007) detection module, they were used at recommended dilution. The prepared plates and capillaries were then loaded onto the JESS, and sample run at the defaults settings. The expression was determined by the area under the curve using Compass Software v 6.1 (ProteinSimple).

### In-Vitro Analysis of Soluble PD-LI Transcript Using RT-qPCR

To determine RNA expression of soluble transcript, cells were lysed and RNA isolated using the RNAeasy Kit (Qiagen, Cat #74136). This was then followed by RNA reverse transcription into cDNA using QuantiNova Reverse Transcription Kit (Qiagen, Cat #205413). Primers for each gene were designed using PrimerQuest™ (Integrated DNA Technologies) and ordered at 100 uM concentrations from IDT. The soluble PD-L1 transcript primer was designed to create an amplicon of about 100 bp size crossing the exon 4 and intron 4 junction. The primer sequences are shown in the table below (Table 3):

| Gene | Forward | Reverse |
|---|---|---|
| PD-L1_ | CTGAATTGGTC | GATAGGCAGAC |
| ex4- | ATCCCAGGTAA | ATCATGCTAGG |
| int4 | (SEQ ID NO: 1) | (SEQ ID NO: 2) |
| OX40L | GCTATGAGTCA | CACAAAGATGG |
| | AGGAGTGTAACC | CTGAGGAAGA |
| | (SEQ ID NO: 3) | (SEQ ID NO: 4) |

RT-qPCR was carried out using the qPCRBIO SyGreen MasterMix kit (PCRBiossystems Cat #PB20.14-50) and the Rotor qene Q qPCR machine (Qiagen). Ct values were analysed using the Rotor qene Q Software. Fold change were calculated using the AACt values.

### Results

In the cell line studies, NUC-7738 was converted into the active anti-cancer metabolite 3′-dATP within 6 hours of treatment with an average concentration of 80 pmoles/10⁶ cells that was maintained for at least 24 hours and decreased by approximately 50% by 72 hours. NUC-7738 decreased mRNA by up to 40% in addition to time dependent decrease of sPD-L1 expression in the media supernatant by up to 3-fold. NUC-7738 also reduced ExoPD-L1 protein by 50%. NUC-7738 did not alter cell surface PD-L1 expression.

Preliminary studies in serum from 4 patients treated with NUC-7738 showed reductions in Exo-PD-L1 of ≤50% compared to pre-treatment levels.

### Example 1

FIG. 1A and FIG. 1B show that NUC-7738 reduces levels of soluble PD-L1 in both lung and melanoma cell lines, supporting the position that NUC-7738 reduces the amount of soluble PD-L1 present in the tumour microenvironment: reduced levels of sPDL1 are known to enhance anti-PD1 and other immune checkpoint therapy.

### Example 2

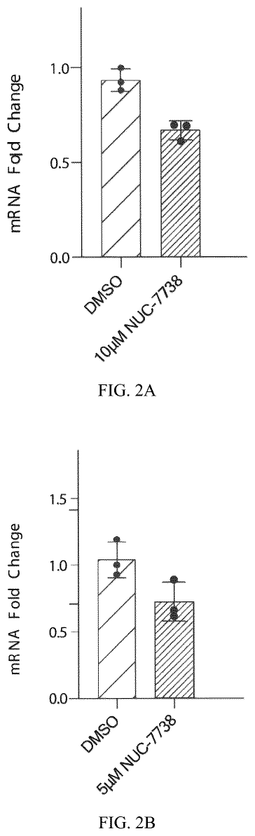

FIG. 2A and FIG. 2B demonstrate that NUC-7738 reduces the amount of transcript of soluble PD-L1 transcript isoform in lung and melanoma cell lines, which supports the premise that the agent acts on tumour cells producing the soluble PD-L1 protein.

### Example 3

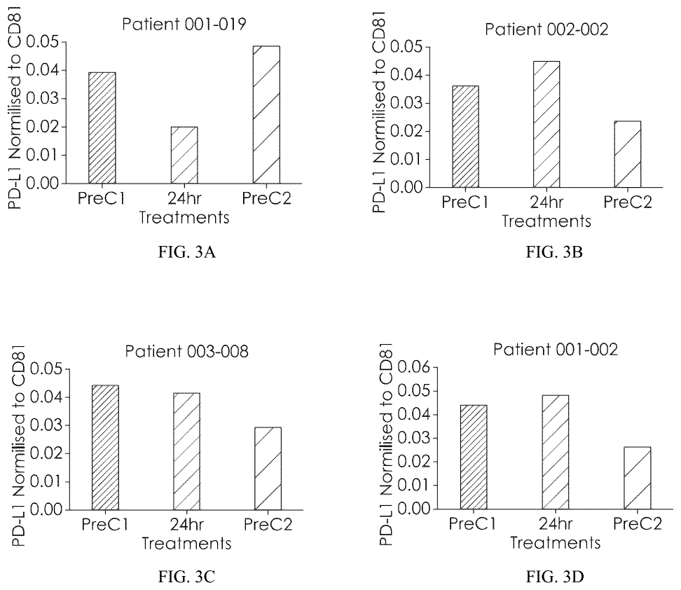

Table 4 (below) and FIG. 3A, FIG. 3B, FIG. 3C, and FIG. 3D show that NUC-7738 reduces levels of exosomal PD-L1 protein in plasma.

| | PD-L1/CD81 values | | |
|---|---|---|---|
| Patient ID | Pre C1 | 24 h Post C1 | Pre C2 |
| 001-002 | 0.044 | 0.047 | 0.025 |
| 002-002 | 0.035 | 0.043 | 0.023 |
| 003-008 | 0.045 | 0.041 | 0.029 |
| 001-019 | 0.039 | 0.02 | 0.049 |

The ratio of PD-L1 and exosome marker CD81 is reduced 15 days after initial NUC-7738 administration in 3 of 4 patients. In patient 001-019 this reduction is seen within 24 hours. This demonstrates that NUC-7738 is able to reduce the level of exosomal PD-L1 in plasma.

### Example 4

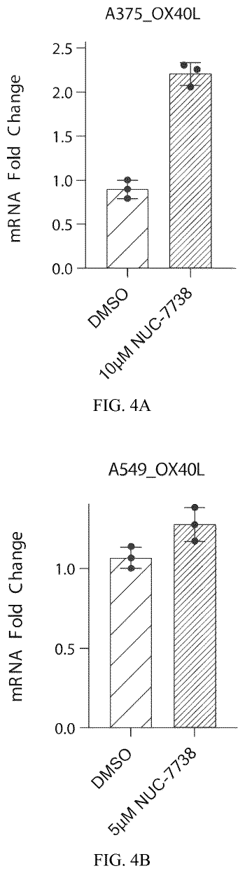

FIG. 4A and FIG. 4B demonstrate that NUC-7738 increased the level of mRNA coding for OX40L in both melanoma and lung cell lines.

### Conclusions:

NUC-7738 reduces secreted forms of PD-L1 whilst having no effect on cell surface protein levels. In vitro data was validated in the clinical setting whereby ExoPD-L1 was reduced in plasma samples from patients treated with NUC-7738. These findings indicate that NUC-7738 has the potential to act as an immune sensitiser and restore T-cell function to promote an anti-cancer immune response. NUC-7738 in combination with PD-(L) 1 pathway inhibitors may offer a promising treatment option, even in patients who have experienced therapeutic resistance to immune checkpoint inhibitor treatment or other reasons for failure of prior immune checkpoint inhibitor treatment (e.g. intolerance or toxicity).

### Example 5—Clinical Study of Combination of NUC-7738 and Pembrolizumab in Patients with Cutaneous Melanoma

A Phase II study, NuTide: 701 expansion, will be conducted to assess the safety, pharmacokinetics and clinical activity of NUC-7738 given on days 1, 8 and 15 of a 21-day cycle with pembrolizumab given on day 1 of the 21-day cycle, to subjects with cutaneous melanoma. Patients will be eligible to receive multiple cycles until disease progression or unacceptable toxicity. Assessment of safety, tolerability and efficacy will be carried out. Levels of extra-cellular PD-L1 will be assessed before and after treatment. Initially 6-12 patent with cutaneous melanoma will be treated. NUC-7738 and pembrolizumab will be given by intravenous infusion.

### Nuc-7738 Infusion.

From a stock vial of NUC-7738 add the required volume of NUC-7738 into a 500 ml standard saline infusion bag; Slowly invert the bag or use a similar method to produce a homogenous clear solution. Continue mixing until the solution is fully mixed. Slowly infuse the contents of the infusion bag containing saline based NUC-7738 formulation to the patient over a period of about 120 minute period. The infusion can be administered via standard means, for example using a central venous administration device or via a cannula.

### Pembrolizumab Infusion.

Withdraw the required volume from the vial(s) of pembrolizumab (KEYTRUDA) and transfer into an intravenous (IV) bag containing 0.9% Sodium Chloride Injection, USP or 5% Dextrose Injection, USP. Mix the diluted solution by gentle inversion. Do not shake. The final concentration of the diluted solution should be between 1 mg/mL to 10 mg/mL

Administer diluted solution intravenously over about 30 minutes through an intravenous line (this is typically done with an infusion pump) containing a sterile, non-pyrogenic, low-protein binding 0.2 micron to 5 micron in-line or add-on filter.

On day 1, the pembrolizumab is administered directly before the NUC-7738 (although the order does not matter).

Subjects to be treated will have histologically confirmed diagnosis of cutaneous melanoma with measurable disease as per RECIST v1.1 criteria. Most subjects will have progressed on≤2 prior lines of therapy for advanced/metastatic cutaneous melanoma, that may have included 1 prior line of an immunotherapy-containing regimen (either monotherapy or in combination with chemotherapy). Patients who have not progressed but where addition of NUC-7738 to standard pembrolizumab monotherapy may be appropriate are also eligible.

In view of NUC-7738's ability to reduce the amount of soluble and exosomal PD-L1, the inventors expect that the combination of NUC-7738 and pembrolizumab will be more efficacious in treating cutaneous melanoma than pembrolizumab given as monotherapy.

We clam:

1. A method for immunomodulation of an immune checkpoint protein in a human in need thereof comprising administration of an effective amount of a compound of the formula (NUC-7738): 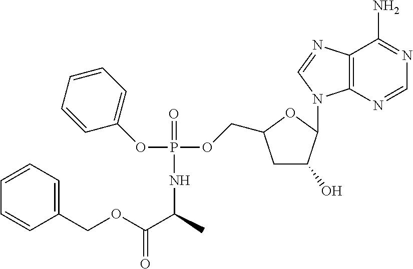 [SMILES: C[C@H](NP(=O)(OCC1C[C@@H](O)C(n2cnc3c(N)ncnc32)O1)Oc1ccccc1)C(=O)OCc1ccccc1]
or a pharmaceutically acceptable salt thereof.

2. The method of claim 1, wherein the immunomodulation comprises decreasing extracellular PD-L1.

3. The method of claim 2, wherein the extracellular PD-L1 is soluble PD-L1.

4. The method of claim 2, wherein the extracellular PD-L1 is exosomal PD-L1.

5. The method of claim 1, wherein the human has a proliferative disease.

6. The method of claim 5, wherein the proliferative disease is cancer.

7. The method of claim 6, wherein the cancer is selected from melanoma, non-small cell lung cancer, urothelial carcinoma, classical Hodgkin lymphoma, head and neck squamous cell carcinoma, renal cell carcinoma, colorectal cancer, and esophageal cancer.

8. The method of claim 7, wherein the cancer is melanoma.

9. A method for inducing adaptive immunity against a proliferative disease in a human in need thereof comprising administering an effective amount of a compound of the formula (NUC-7738): 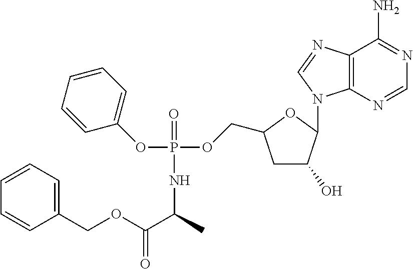 [SMILES: C[C@H](NP(=O)(OCC1C[C@@H](O)C(n2cnc3c(N)ncnc32)O1)Oc1ccccc1)C(=O)OCc1ccccc1]
or a pharmaceutically acceptable salt thereof.

10. The method of claim 9, wherein the proliferative disease is cancer.

11. The method of claim 10, wherein the cancer is melanoma.

12. A method of treating cancer in a human in need thereof comprising administration of a compound of the formula (NUC-7738): 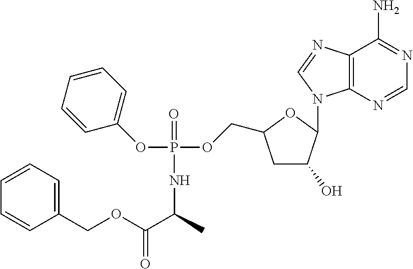 [SMILES: C[C@H](NP(=O)(OCC1C[C@@H](O)C(n2cnc3c(N)ncnc32)O1)Oc1ccccc1)C(=O)OCc1ccccc1]
or a pharmaceutically acceptable salt thereof, in combination with pembrolizumab.

13. The method of claim 12, wherein the cancer is resistant to pembrolizumab.

14. The method of claim 12, wherein the human in need thereof previously received one or more prior treatments of pembrolizumab.

15. The method of claim 12, comprising administering from about 300 mg/m² to about 1600 mg/m² of NUC-7738.

16. The method of claim 12, comprising administering NUC-7738 about once per week.

17. The method of claim 12, comprising administering NUC-7738 for about three weeks.

18. A method for treating a cancer that is resistant to checkpoint inhibitor therapy in a human in need thereof comprising administration of a compound of the formula (NUC-7738): 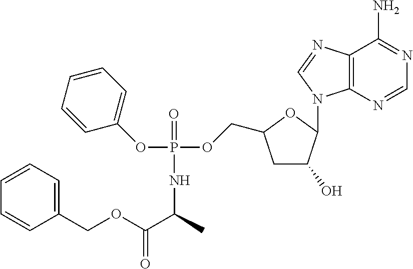 [SMILES: C[C@H](NP(=O)(OCC1C[C@@H](O)C(n2cnc3c(N)ncnc32)O1)Oc1ccccc1)C(=O)OCc1ccccc1]
or a pharmaceutically acceptable salt thereof.

19. The method of claim 18, wherein the cancer is melanoma.

20. The method of claim 19, wherein the melanoma is cutaneous melanoma.
